(19) United States
(12) Patent Application Publication
(10) Pub. No.: US 2024/0382402 A1
(43) Pub. Date: Nov. 21, 2024

(54) COMPOSITION FOR PREVENTING OR REDUCING MICROBES ON A SURFACE

(71) Applicant: Ring-Co Health, LLC, Trafalgar, IN (US)

(72) Inventors: Chad Ringer, Trafalgar, IN (US); Patricia Ringer, Trafalgar, IN (US)

(21) Appl. No.: 18/555,336

(22) PCT Filed: Apr. 13, 2022
(86) PCT No.: PCT/US22/24562

§ 371 Date: Oct. 13, 2023

Related U.S. Application Data
(60) Provisional application No. 63/175,219, filed on Apr. 15, 2021.

(51) Int. Cl. A61K 8/43; A61K 8/34; A61Q 17/00

(52) U.S. Cl. CPC A61K 8/43; A61K 8/34; A61Q 17/005; A61K 2800/591

(57) ABSTRACT

Topical disinfectant compositions for sanitizing skin or other surfaces or increasing the surfaces' resistance to microbe transmission according to certain embodiments include ethyl alcohol, chlorhexidine, an ester-containing moisturizer and water, and optionally also include one or more of a surfactant, a preservative, colloidal silver and hydrogen peroxide. Alternative topical disinfection compositions for sanitizing skin or other surfaces or increasing the surfaces' resistance to microbe transmission include hydrogen peroxide, chlorhexidine, an ester-containing moisturizer and water, and optionally also include one or more of a surfactant, a preservative and colloidal silver.

### CROSS-REFERENCE TO RELATED APPLICATION

The present application claims priority to U.S. Provisional Patent Application No. 63/175,219, which was filed Apr. 15, 2021, the entire content of which is incorporated by reference herein.

### BACKGROUND

A great need exists for additional ways to inhibit transmission of infectious diseases from one person to another, including a need for effective compositions that prevent or reduce survival or proliferation of microbes on a surface, such as on hands or other skin surfaces and on surfaces typically touched or otherwise contacted by multiple persons in ordinary use. A need also exists for such compositions that are user friendly to enhance widespread use. These needs are especially pronounced at the present point in time in view of the widespread health and safety concerns that have arisen in the wake of the worldwide COVID-19 pandemic. The present disclosure addresses these needs.

### SUMMARY

According to an embodiment, a topical disinfectant composition for sanitizing a surface or increasing the surface's resistance to microbe transmission comprises ethyl alcohol, chlorhexidine, an ester-containing moisturizer and water. In some embodiments, the ester-containing moisturizer comprises gluconolactone. In some embodiments, the composition further comprises a surfactant. In some embodiments, the surfactant comprises lauramine oxide. In some embodiments, the composition further comprises a preservative. In some embodiments, the composition further comprises colloidal silver.

In some embodiments, the composition further comprises hydrogen peroxide. In some embodiments, a composition comprises hydrogen peroxide in place of, and instead of, ethyl alcohol.

According to another embodiment, a method for sanitizing a surface or increasing the surface's resistance to microbe transmission includes providing a composition comprising ethyl alcohol, chlorhexidine, an ester-containing moisturizer, and water; selecting a surface to be sanitized; contacting the composition with the surface; and either permitting the ethyl alcohol and the water to evaporate or wiping the surface with a drying cloth. In some embodiments, the surface to be sanitized comprises a human skin area. In some embodiments, the surface to be sanitized comprises a person's hand. In some embodiments, the surface to be sanitized comprises a member selected from the group consisting of skin, a wrestling mat, a basketball, volleyball, shotput or other article of athletic equipment a computer keyboard, a chair, a hand rail, a door, a door handle, a refrigerator handle, a countertop, a gaming controller, a writing utensil and a nursery or preschool toy. In some embodiments, the ester-containing moisturizer comprises gluconolactone. In some embodiments, the composition further comprises a surfactant. In some embodiments, the surfactant comprises lauramine oxide. In some embodiments, the composition further comprises a preservative. In some embodiments, the composition further comprises colloidal silver.

According to another embodiment, a method for making a topical disinfectant composition for sanitizing a surface or increasing the surface's resistance to microbe transmission includes providing a first quantity of water; dissolving a second quantity of an ester-containing moisturizer into the first quantity of water while agitating the water to provide a solution; mixing a third quantity of ethyl alcohol into the first solution while continuing to agitate the solution to provide a mixture; adding a fourth quantity of chlorhexidine to the mixture to provide a composition; and continuing to agitate the composition for a period of time. In some embodiments, the water comprises distilled water. In some embodiments, the ester-containing moisturizer comprises gluconolactone.

In some embodiments, the agitating comprises an action selected from the group consisting of stirring, shaking and combinations thereof.

In some embodiments, the period of time is from about 30 seconds to about 30 minutes. In some embodiments, the period of time is from about 2 minutes to about 15 minutes.

In some embodiments, the first quantity, the second quantity, the third quantity and the fourth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight and the chlorhexidine comprises from about 0.1 to about 4% of the composition by weight.

In some embodiments, the method further includes diffusing a fifth quantity of a surfactant into the water or into the solution. In some embodiments, the surfactant comprises lauramine oxide.

In some embodiments, the first quantity, the second quantity, the third quantity, the fourth quantity and the fifth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight and the surfactant comprises from about 0.1 to about 2% of the composition by weight.

In some embodiments, the method further includes dissolving a sixth quantity of sodium benzoate into the water or into the solution. In some embodiments, the first quantity, the second quantity, the third quantity, the fourth quantity, the fifth quantity and the sixth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight, the surfactant comprises from 0 to about 2% of the composition by weight and the preservative comprises from about 0.1 to about 2% of the composition by weight.

In some embodiments, the method further includes dispersing a seventh quantity of colloidal silver into the water, into the solution, into the mixture or into the composition. In some embodiments, the first quantity, the second quantity, the third quantity, the fourth quantity, the fifth quantity, the sixth quantity and the seventh quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight, the surfactant comprises from 0 to about 2% of the composition by weight, the preservative comprises from 0 to about 2% of the composition by weight and the colloidal silver comprises from about 0.1 to about 2% of the composition by weight.

In some embodiments, the method further includes diffusing an eighth quantity of hydrogen peroxide into the water or into the solution. In some embodiments, the first quantity, the second quantity, the third quantity, the fourth quantity and the eighth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight and the hydrogen peroxide comprises from about 0.05 to about 30% of the composition by weight.

According to another embodiment, a method for making a topical disinfectant composition for sanitizing a surface or increasing the surface's resistance to microbe transmission includes providing a first quantity of water; dissolving a second quantity of an ester-containing moisturizer into the first quantity of water while agitating the water to provide a solution; mixing a third quantity of hydrogen peroxide into the first solution while continuing to agitate the solution to provide a mixture; adding a fourth quantity of chlorhexidine to the mixture to provide a composition; and continuing to agitate the composition for a period of time. In some embodiments, the water comprises distilled water. In some embodiments, the ester-containing moisturizer comprises gluconolactone.

In some embodiments, the first quantity, the second quantity, the third quantity and the fourth quantity are selected such that the method produces a composition in which the water comprises from about 15 to about 98% of the composition by weight, the ester-containing moisturizer comprises from about 0.1 to about 5% of the composition by weight, the hydrogen peroxide comprises from about 0.05 to about 30% of the composition by weight and the chlorhexidine comprises from about 0.1 to about 4% of the composition by weight.

This summary is not intended to identify key or essential features of the claimed subject matter, nor is it intended to be used as an aid in limiting the scope of the claimed subject matter. Further embodiments, forms, features, and aspects of the present application shall become apparent from the description and figures provided herewith.

### BRIEF DESCRIPTION OF THE DRAWINGS

The concepts described herein are illustrated by way of example and not by way of limitation in the accompanying figures.

FIG. 1 depicts test results for an evaluation of microorganism growth on an agar plate labeled as “pre-wash LEFT thumb” as described in Example 4

FIG. 2 depicts test results for an evaluation of microorganism growth on an agar plate labeled as “post-wash LEFT thumb” as described in Example 4.

FIG. 3 depicts test results for an evaluation of microorganism growth on an agar plate labeled as “pre-wash RIGHT thumb” as described in Example 4.

FIG. 4 depicts test results for an evaluation of microorganism growth on an agar plate labeled as “immediately after Compel™ RIGHT thumb” as described in Example 4.

FIG. 5 depicts test results for an evaluation of microorganism growth on an agar plate labeled as “3 hours after Compel™ RIGHT thumb” as described in Example 4.

FIG. 6 depicts test results for an evaluation of microorganism growth on an agar plate labeled as “keyboard prior to treatment” as described in Example 4.

FIG. 7 depicts test results for an evaluation of microorganism growth on an agar plate labeled as “keyboard immediately after Compel™” as described in Example 4.

FIG. 8 depicts test results for an evaluation of microorganism growth on an agar plate labeled as “keyboard 3 hours after Compel™” as described in Example 4.

FIG. 9 depicts test results for an evaluation of microorganism growth on an agar plate labeled as “office chair prior to treatment” as described in Example 4.

FIG. 10 depicts test results for an evaluation of microorganism growth on an agar plate labeled as “office chair 3 hours after Compel™” as described in Example 4.

FIG. 11 depicts test results for an evaluation of microorganism growth on an agar plate labeled as “refrigerator handle before treatment” as described in Example 5.

FIG. 12 depicts test results for an evaluation of microorganism growth on an agar plate labeled as “PS4 controller before treatment” as described in Example 5.

FIG. 13 depicts test results for an evaluation of microorganism growth on an agar plate labeled as “refrigerator handle-8 hours post Compel™” as described in Example 5.

FIG. 14 depicts test results for an evaluation of microorganism growth on an agar plate labeled as “PS4 controller-8 hours post Compel™” as described in Example 5.

FIG. 15 depicts test results for an evaluation of microorganism growth on an agar plate labeled as “refrigerator handle-12 hours post Compel™” as described in Example 5.

FIG. 16 depicts test results for an evaluation of microorganism growth on an agar plate labeled as “PS4 controller-12 hours post Compel™” as described in Example 5.

FIG. 17 depicts test results for an evaluation of microorganism growth on an agar plate labeled as “refrigerator handle-24 hours post Compel™” as described in Example 5.

FIG. 18 depicts test results for an evaluation of microorganism growth on an agar plate labeled as “PS4 controller-24 hours post Compel™” as described in Example 5.

FIG. 19 depicts test results for an evaluation of microorganism growth on an agar plate labeled as “left thumb-4 hours post Compel™—no touch” as described in Example 5.

FIG. 20 depicts test results for an evaluation of microorganism growth on an agar plate labeled as “right thumb-4 hours post Compel™—touch” as described in Example 5.

FIG. 21 depicts test results for an evaluation of microorganism growth on an agar plate labeled as “wrestling mat area A prior to treatment” as described in Example 6.

FIG. 22 depicts test results for an evaluation of microorganism growth on an agar plate labeled as “wrestling mat area B immediately after MadaCide” as described in Example 6.

FIG. 23 depicts test results for an evaluation of microorganism growth on an agar plate labeled as “wrestling mat area A immediately after Compel™ treatment” as described in Example 6.

FIG. 24 depicts test results for an evaluation of microorganism growth on an agar plate labeled as “wrestling mat area A 1.5 hours after Compel™ treatment” as described in Example 6.

FIG. 25 depicts test results for an evaluation of microorganism growth on an agar plate labeled as “wrestling mat area C 2 days after Compel™ treatment” as described in Example 6.

FIG. 26 depicts test results for an evaluation of microorganism growth on an agar plate labeled as “wrestler A prior to treatment” as described in Example 7.

FIG. 27 depicts test results for an evaluation of microorganism growth on an agar plate labeled as “wrestler A post Compel™ and full practice” as described in Example 7.

FIG. 28 depicts test results for an evaluation of microorganism growth on an agar plate labeled as “wrestler B prior to treatment” as described in Example 7.

FIG. 29 depicts test results for an evaluation of microorganism growth on an agar plate labeled as “wrestler B post hand sanitizer and full practice” as described in Example 7.

FIG. 30 depicts test results for an evaluation of microorganism growth on an agar plate labeled as “upper wrestling room mat prior to treatment” as described in Example 8.

FIG. 31 depicts test results for an evaluation of microorganism growth on an agar plate labeled as “upper wresting room mat immediately after Compel™ treatment” as described in Example 8.

FIG. 32 depicts test results for an evaluation of microorganism growth on an agar plate labeled as “wrestling scale touch pad prior to treatment” as described in Example 8.

FIG. 33 depicts test results for an evaluation of microorganism growth on an agar plate labeled as “wrestling scale touch pad immediately after Compel™ treatment” as described in Example 8.

FIG. 34 depicts test results for an evaluation of microorganism growth on an agar plate labeled as “lower room mat area A at 24 hours post Compel™ treatment” as described in Example 8.

FIG. 35 depicts test results for an evaluation of microorganism growth on an agar plate labeled as “lower room mat area A at 1 week post Compel™ treatment” as described in Example 8.

FIG. 36 depicts test results for an evaluation of microorganism growth on an agar plate labeled as “upper room mat 1 week post Compel™ treatment” as described in Example 8.

FIG. 37 depicts test results for an evaluation of microorganism growth on an agar plate labeled as “wrestling scale foot pad pre-treatment” as described in Example 8.

FIG. 38 depicts test results for an evaluation of microorganism growth on an agar plate labeled as “pre-treatment—Church B Front door (48 hrs)” as described in Example 9.

FIG. 39 depicts test results for an evaluation of microorganism growth on an agar plate labeled as “immediately post Compel™—Church B Front door (48 hrs)” as described in Example 9.

FIG. 40 depicts test results for an evaluation of microorganism growth on an agar plate labeled as “pre-treatment—Church B Bathroom door (48 hrs)” as described in Example 9.

FIG. 41 depicts test results for an evaluation of microorganism growth on an agar plate labeled as “immediately post Compel™—Church B Bathroom door (48 hrs)” as described in Example 9.

FIG. 42 depicts test results for an evaluation of microorganism growth on an agar plate labeled as “pre-treatment—Church B Hand rail (48 hrs)” as described in Example 9.

FIG. 43 depicts test results for an evaluation of microorganism growth on an agar plate labeled as “immediately post Compel™—Church B Hand rail (48 hrs)” as described in Example 9.

FIG. 44 depicts test results for an evaluation of microorganism growth on an agar plate labeled as “pre-treatment—Church B Check-in (48 hrs)” as described in Example 9.

FIG. 45 depicts test results for an evaluation of microorganism growth on an agar plate labeled as “immediately post Compel™—Church B Check-in (48 hrs)” as described in Example 9.

FIG. 46 depicts test results for an evaluation of microorganism growth on an agar plate labeled as “pre-treatment—Church B Café (48 hrs)” as described in Example 9.

FIG. 47 depicts test results for an evaluation of microorganism growth on an agar plate labeled as “immediately post Compel™—Church B Café (48 hrs)” as described in Example 9.

FIG. 48 depicts test results for an evaluation of microorganism growth on an agar plate labeled as “pre-treatment—Church A Front door (48 hrs)” as described in Example 9.

FIG. 49 depicts test results for an evaluation of microorganism growth on an agar plate labeled as “immediately post Compel™—Church A Front door (48 hrs)” as described in Example 9.

FIG. 50 depicts test results for an evaluation of microorganism growth on an agar plate labeled as “pre-treatment—Church A Bathroom entry (48 hrs)” as described in Example 9.

FIG. 51 depicts test results for an evaluation of microorganism growth on an agar plate labeled as “immediately post Compel™—Church A Bathroom door (48 hrs)” as described in Example 9.

FIG. 52 depicts test results for an evaluation of microorganism growth on an agar plate labeled as “pre-treatment—Church A Hand rail (48 hrs)” as described in Example 9.

FIG. 53 depicts test results for an evaluation of microorganism growth on an agar plate labeled as “immediately post Compel™—Church A Hand rail (48 hrs)” as described in Example 9.

FIG. 54 depicts test results for an evaluation of microorganism growth on an agar plate labeled as “pre-treatment—Church A Front desk (48 hrs)” as described in Example 9.

FIG. 55 depicts test results for an evaluation of microorganism growth on an agar plate labeled as “immediately post Compel™—Church A Front desk (48 hrs)” as described in Example 9.

FIG. 56 depicts test results for an evaluation of microorganism growth on an agar plate labeled as “pre-treatment—Church A 2s and 3s table (48 hrs)” as described in Example 9.

FIG. 57 depicts test results for an evaluation of microorganism growth on an agar plate labeled as “immediately post Compel™—Church A 2s and 3s table (48 hrs)” as described in Example 9.

FIG. 58 depicts test results for an evaluation of microorganism growth on an agar plate labeled as “pre-treatment—Church B Front door (96 hrs)” as described in Example 9.

FIG. 59 depicts test results for an evaluation of microorganism growth on an agar plate labeled as “immediately post Compel™—Church B Front door (96 hrs)” as described in Example 9.

FIG. 60 depicts test results for an evaluation of microorganism growth on an agar plate labeled as “pre-treatment—Church B Bathroom door (96 hrs)” as described in Example 9.

FIG. 61 depicts test results for an evaluation of microorganism growth on an agar plate labeled as “immediately post Compel™—Church B Bathroom door (96 hrs)” as described in Example 9.

FIG. 62 depicts test results for an evaluation of microorganism growth on an agar plate labeled as “pre-treatment—Church B Hand rail (96 hrs)” as described in Example 9.

FIG. 63 depicts test results for an evaluation of microorganism growth on an agar plate labeled as “immediately post Compel™—Church B Hand rail (96 hrs)” as described in Example 9.

FIG. 64 depicts test results for an evaluation of microorganism growth on an agar plate labeled as “pre-treatment—Church B Check-in (96 hrs)” as described in Example 9.

FIG. 65 depicts test results for an evaluation of microorganism growth on an agar plate labeled as “immediately post Compel™—Church B Check-in (96 hrs)” as described in Example 9.

FIG. 66 depicts test results for an evaluation of microorganism growth on an agar plate labeled as “pre-treatment—Church B Café (96 hrs)” as described in Example 9.

FIG. 67 depicts test results for an evaluation of microorganism growth on an agar plate labeled as “immediately post Compel™—Church B Café (96 hrs)” as described in Example 9.

FIG. 68 depicts test results for an evaluation of microorganism growth on an agar plate labeled as “pre-treatment—Church A Front door (96 hrs)” as described in Example 9.

FIG. 69 depicts test results for an evaluation of microorganism growth on an agar plate labeled as “immediately post Compel™—Church A Front door (96 hrs)” as described in Example 9.

FIG. 70 depicts test results for an evaluation of microorganism growth on an agar plate labeled as “Pre-treatment—Church A Bathroom entry (96 hrs)” as described in Example 9.

FIG. 71 depicts test results for an evaluation of microorganism growth on an agar plate labeled as “Immediately post Compel™—Church A Bathroom door (96 hrs)” as described in Example 9.

FIG. 72 depicts test results for an evaluation of microorganism growth on an agar plate labeled as “Pre-treatment—Church A Hand rail (96 hrs)” as described in Example 9.

FIG. 73 depicts test results for an evaluation of microorganism growth on an agar plate labeled as “Immediately post Compel™—Church A Hand rail (96 hrs)” as described in Example 9.

FIG. 74 depicts test results for an evaluation of microorganism growth on an agar plate labeled as “Pre-treatment—Church A Front desk (96 hrs)” as described in Example 9.

FIG. 75 depicts test results for an evaluation of microorganism growth on an agar plate labeled as “Immediately post Compel™—Church A Front desk (96 hrs)” as described in Example 9.

FIG. 76 depicts test results for an evaluation of microorganism growth on an agar plate labeled as “Pre-treatment—Church A 2s and 3s table (96 hrs)” as described in Example 9.

FIG. 77 depicts test results for an evaluation of microorganism growth on an agar plate labeled as “Immediately post Compel™—Church A 2s and 3s table (96 hrs)” as described in Example 9.

FIG. 78 depicts test results for an evaluation of microorganism growth on an agar plate labeled as “1 week post Compel™—Church B Front door (96 hrs)” as described in Example 9.

FIG. 79 depicts test results for an evaluation of microorganism growth on an agar plate labeled as “1 week post Compel™—Church B Bathroom door (96 hrs)” as described in Example 9.

FIG. 80 depicts test results for an evaluation of microorganism growth on an agar plate labeled as “1 week post Compel™—Church B Hand rail (96 hrs)” as described in Example 9.

FIG. 81 depicts test results for an evaluation of microorganism growth on an agar plate labeled as “1 week post Compel™—Church B Children area check-in (96 hrs)” as described in Example 9.

FIG. 82 depicts test results for an evaluation of microorganism growth on an agar plate labeled as “1 week post Compel™—Church B Café countertop (96 hrs)” as described in Example 9.

FIG. 83 depicts test results for an evaluation of microorganism growth on an agar plate labeled as “1 week post Compel™—Church A Front entry door (96 hrs)” as described in Example 9.

FIG. 84 depicts test results for an evaluation of microorganism growth on an agar plate labeled as “1 week post Compel™—Church A Bathroom door (96 hrs)” as described in Example 9.

FIG. 85 depicts test results for an evaluation of microorganism growth on an agar plate labeled as “1 week post Compel™—Church A Hand rail (96 hrs, broken plate)” as described in Example 9.

FIG. 86 depicts test results for an evaluation of microorganism growth on an agar plate labeled as “1 week post Compel™—Church A Front desk (96 hrs, check-in)” as described in Example 9.

FIG. 87 depicts test results for an evaluation of microorganism growth on an agar plate labeled as “1 week post Compel™—Church A 2s and 3s tabletop (96 hrs)” as described in Example 9.

### DETAILED DESCRIPTION

In general, this disclosure involves antimicrobial compositions that are used to topically sanitize skin areas and/or other surfaces.

Although the concepts of the present disclosure are susceptible to various modifications and alternative forms, specific embodiments are shown by way of example and described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims. References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1.5 to 4, from 1 to 5.23, from 2 to 4, from 2.7 to 6, from 3.65 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

In one aspect, this disclosure provides a topical antimicrobial or disinfectant composition for sanitizing a surface or increasing the surface's resistance to microbe transmission. In one embodiment, a composition includes ethyl alcohol, chlorhexidine, an ester-containing moisturizer and water. In some embodiments, the ester-containing moisturizer comprises gluconolactone. In some embodiments, the composition further comprises a surfactant such as, for example and without limitation, lauramine oxide. In some embodiments, the composition further comprises a preservative such as, for example and without limitation, sodium benzoate. In some embodiments, the composition further comprises at least one beneficial additive such as, for example and without limitation, colloidal silver.

In some embodiments, the composition further comprises hydrogen peroxide. In some embodiments, the hydrogen peroxide comprises from about 0.05 to about 30% of the composition by weight. In some embodiments, a composition comprises hydrogen peroxide in place of, and instead of, ethyl alcohol. For purposes of efficiency in the present disclosure, most embodiments are described as including ethyl alcohol; however, the description of each such embodiment is expressly intended to constitute a description of the variation of such embodiment in which hydrogen peroxide is substituted for ethyl alcohol.

In some embodiments, the ethyl alcohol included in the composition is provided in the form of 190-proof undenatured ethyl alcohol. In other embodiments, other sources of ethyl alcohol are used. In some embodiments, the chlorhexidine included in the composition is provided in the form of chlorhexidine gluconate. For example, a 20% chlorhexidine gluconate solution is a suitable starting material for making such composition. In other embodiments, other sources of chlorhexidine are used. In some embodiments, the water included in the composition is provided in the form of distilled water. In other embodiments, other sources of water are used. While purified water, filtered water, spring water, or even tap water can be used, uses of water from such sources can produce undesirable particulates in the composition.

In some embodiments, the ethyl alcohol comprises from about 1 to about 85% of the composition by weight, the chlorhexidine comprises from about 0.1 to about 4% of the composition by weight, the ester-containing moisturizer comprises from about 0.1 to about 5% of the composition by weight and the water comprises from about 15 to about 98% of the composition by weight. In some embodiments, the ester-containing moisturizer comprises gluconolactone. In some embodiments, the composition further comprises a surfactant in an amount of from about 0.1 to about 2% of the composition by weight. In some embodiments, the surfactant comprises lauramine oxide. In some embodiments, the composition further comprises a preservative in an amount of from about 0.1 to about 2% of the composition by weight. In some embodiments, the preservative comprises sodium benzoate. In some embodiments, the composition further comprises colloidal silver in an amount of from about 0.1 to about 2% of the composition by weight.

In some embodiments that include both an ester-containing moisturizer and a preservative, the ester-containing moisturizer and the preservative in the composition can be provided in the form of a combined gluconolactone/sodium benzoate product (also referred to herein as “GSB”), examples of which are readily available commercially. One example of a suitable GSB for use in making a composition according to this disclosure is the Lotioncrafter® NeoDefend™ product produced by Lotioncrafter LLC (Olga, Washington).

In another aspect of the disclosure, methods are provided in which a composition as described herein is used as a topical antimicrobial treatment. A topical antimicrobial composition can be used, for example, to sanitize one's hands or other skin area or to sanitize or clean other surfaces, devices or the like. In one form of the disclosure, there is provided a method for sanitizing a surface or increasing the surface's resistance to microbe transmission that includes: (i) providing a composition comprising ethyl alcohol, chlorhexidine, an ester-containing moisturizer, and water as described herein; selecting a surface to be sanitized; contacting the composition with the surface; and either permitting the ethyl alcohol and the water to evaporate or wiping the surface with a drying cloth. The surface can be, for example, a human skin area such as a person's hands, a wrestling mat, a basketball, volleyball, shotput or other article of athletic equipment, a computer keyboard, a chair, a hand rail, a door, a door handle, a refrigerator handle, a countertop, a gaming controller, a writing utensil or a nursery or preschool toy. Sanitizing can be achieved by soaking, rinsing, wiping rubbing or swabbing one's hands or other surface to be treated in or with a composition described herein and either permitting the ethyl alcohol and water to evaporate or wiping the surface with a drying cloth.

One advantage of compositions described herein is that no rinsing is required after application of the composition to a surface, which permits use of the compositions by application to hands or other surfaces in environments where water for rinsing is not available or is not convenient. Another advantage of such compositions, however, when used to clean floors, hand rails and other such surfaces or facilities, is that the composition has excellent effect even when a drying cloth is used to wipe down a surface immediately following application of the composition to the surface. Many industrial cleaners used in the prior art for cleaning/sanitizing such surfaces require that the surface remain wetted with the prior art composition for several minutes before wiping, which causes significant inefficiencies and challenges that are avoided by compositions of the present disclosure.

In another aspect, the present disclosure provides a method for making a topical disinfectant composition for sanitizing a surface or increasing the surface's resistance to microbe transmission. In one embodiment, the method includes: (i) providing a first quantity of water; (ii) dissolving a second quantity of an ester-containing moisturizer into the first quantity of water while agitating the water to provide a solution; (iii) mixing a third quantity of ethyl alcohol into the first solution while continuing to agitate the solution to provide a mixture; (iv) adding a fourth quantity of chlorhexidine to the mixture to provide a composition; and (v) continuing to agitate the composition for a period of time. In some embodiments, the water comprises distilled water. In some embodiments, the agitating can be achieved by stirring, shaking or combinations thereof. In some embodiments, the period of time is from about 30 seconds to about 30 minutes. In some embodiments, the period of time is from about 2 minutes to about 15 minutes. In some embodiments, the first quantity, the second quantity, the third quantity and the fourth quantity are selected such that the method produces a composition in which the water comprises from about 15 to about 98% of the composition by weight, the ester-containing moisturizer comprises from about 0.2 to about 6% of the composition by weight, the ethyl alcohol comprises from about 1 to about 85% of the composition by weight and the chlorhexidine comprises from about 0.1 to about 4% of the composition by weight.

In some embodiments, the method further includes diffusing a fifth quantity of a surfactant into the water or into the solution. In some embodiments, the first quantity, the second quantity, the third quantity, the fourth quantity and the fifth quantity are selected such that the method produces a composition in which the water comprises from about 15 to about 98% of the composition by weight, the ester-containing moisturizer comprises from about 0.1 to about 4% of the composition by weight, the ethyl alcohol comprises from about 1 to about 85% of the composition by weight, the chlorhexidine comprises from about 0.1 to about 4% of the composition by weight and the surfactant comprises from about 0.1 to about 2% of the composition by weight.

In some embodiments, any of the embodiments described above further includes dissolving a sixth quantity of a preservative, such sodium benzoate, into the water or into the solution. In some embodiments, the first quantity, the second quantity, the third quantity, the fourth quantity and the sixth quantity are selected such that the method produces a composition in which the water comprises from about 15 to about 98% of the composition by weight, the ester-containing moisturizer comprises from about 0.1 to about 4% of the composition by weight, the ethyl alcohol comprises from about 1 to about 85% of the composition by weight, the chlorhexidine comprises from about 0.1 to about 4% of the composition by weight and the preservative comprises from about 0.1 to about 2% of the composition by weight. In some embodiments, the first quantity, the second quantity, the third quantity, the fourth quantity, the fifth quantity and the sixth quantity are selected such that the method produces a composition in which the water comprises from about 15 to about 98% of the composition by weight, the ester-containing moisturizer comprises from about 0.1 to about 2% of the composition by weight, the ethyl alcohol comprises from about 1 to about 85% of the composition by weight, the chlorhexidine comprises from about 0.1 to about 4% of the composition by weight, the surfactant comprises from about 0 to about 4% of the composition by weight and the preservative comprises from about 0 to about 4% of the composition by weight.

In some embodiments, any of the embodiments described above further includes dispersing a seventh quantity of colloidal silver into the water, into the solution, into the mixture or into the composition. In some embodiments, the first quantity, the second quantity, the third quantity, the fourth quantity and the seventh quantity are selected such that the method produces a composition in which the water comprises from about 15 to about 98% of the composition by weight, the ester-containing moisturizer comprises from about 0.1 to about 4% of the composition by weight, the ethyl alcohol comprises from about 1 to about 85% of the composition by weight, the chlorhexidine comprises from about 0.1 to about 4% of the composition by weight and the colloidal silver comprises from about 0.1 to about 4% of the composition by weight. In some embodiments, the first quantity, the second quantity, the third quantity, the fourth quantity, the fifth quantity, the sixth quantity and the seventh quantity are selected such that the method produces a composition in which the water comprises from about 15 to about 98% of the composition by weight, the ester-containing moisturizer comprises from about 0.1 to about 4% of the composition by weight, the ethyl alcohol comprises from about 1 to about 85% of the composition by weight, the chlorhexidine comprises from about 0.1 to about 4% of the composition by weight, the surfactant comprises from about 0 to about 4% of the composition by weight, the preservative comprises from about 0 to about 4% of the composition by weight and the colloidal silver comprises from about 0 to about 4% of the composition by weight.

Further reference is made to the following experimental examples.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention. Changes therein and other uses which are encompassed within the spirit of the invention as defined by the scope of the claims will occur to those skilled in the art.

### Example 1

A sample of a test composition as described in the present disclosure (referred to herein as “Compel™”) for preventing or reducing microbes on a surface was made at room temperature by mixing 779 ml of distilled water with 28.4 grams of NeoDefend™ GSB under constant stirring using a magnetic stirrer until the GSB was fully dissolved (about 5 minutes). According to available product literature, the NeoDefend™ GSB product was composed of CAS #90-80-2 (74.25%), CAS #532-321 (24.75%) and CAS #299-28-5 (1.0%). As stirring continued, 9.5 ml of lauramine oxide (CAS #1643-20-5) was added to the solution and mixed until the lauramine oxide was fully dissolved or dispersed in the solution (about 5 minutes), and then 2.59 liters of 190-proof ethyl alcohol (CAS #64-17-5) was added to the solution as the solution continued to be stirred. When the mixture was thoroughly mixed (after about 5 minutes), 378.5 ml of a 20% solution of chlorhexidine gluconate (in water; CAS #18472-51-1) was added to the mixture under continuous stirring until all cloudiness was gone and the liquid returned to clear (about 5 to 10 minutes). The pH was then checked to confirm that pH of the formulation is between 6 and 8. The sample as made was composed of approximately 32% water, 0.75% GSB, 0.25% lauramine oxide, 65.0% ethyl alcohol and 2.0% chlorhexidine gluconate.

### Example 2

A quantity of Compel™ with enhanced skin moisturizing effectiveness was made at room temperature by mixing 703 ml of distilled water with 37.9 grams of NeoDefend™ GSB under constant stirring using a magnetic stirrer until the GSB was fully dissolved (about 5 minutes). According to available product literature, the NeoDefend™ GSB product was composed of CAS #90-80-2 (74.25%), CAS #532-321 (24.75%) and CAS #299-28-5 (1.0%). As stirring continued, 75.7 ml of lauramine oxide (CAS #1643-20-5) was added to the solution and mixed until the lauramine oxide was fully dissolved or dispersed in the solution (about 5 minutes), and then 2.59 liters of 190-proof ethyl alcohol (CAS #64-17-5) was added to the solution as the solution continued to be stirred. When the mixture was thoroughly mixed (after about 5 minutes), 378.5 ml of a 20% solution of chlorhexidine gluconate (in water; CAS #18472-51-1) was added to the mixture under continuous stirring until all cloudiness was gone and the liquid returned to clear (about 5 to 10 minutes). The pH was then checked to confirm that pH of the formulation is between 6 and 8. The sample as made was composed of approximately 30% water, 1.0% GSB. 2.0% lauramine oxide, 65.0% ethyl alcohol and 2.0% chlorhexidine gluconate.

### Example 3

The Compel™ samples made as described in Example 1 and Example 2 were tested for microbe resistance efficacy in several environments and compared to a variety of alternative products and practices for killing or containing microbe contamination. More specifically, the ability of Compel™ to kill or reduce growth of microorganisms on surfaces, notably hands and various fomites, including furniture, door handles, railings, wrestling mats, and various other high contact areas was assessed. For all tests, general purpose media were used to demonstrate presence of viable microbes, as these plates support a wide variety of non-fastidious organisms, including bacteria, mold, and fungi. While species determination was not performed, and thus claims cannot be made that these tests definitively prevented or reduced contamination of a specific pathogen (e.g. bacteria such as MRSA, various viruses including SARS-COV-2, or fungi that cause ringworm), they can be interpreted as reducing the total number of viable microorganisms on these surfaces, which could include these pathogens.

### General Methods

Non-selective, general purpose media (Nutrient Agar or Tryptic Soy Agar, obtained from Evviva Sciences, Fremont, CA, or Hardy Diagnostics, Santa Maria, CA) was stored at 4° C. Plates were acclimated to room temperature for 5-10 min prior to testing. Unless noted, for testing skin the surface of either the left or right thumb was used by directly pressing a thumb onto the surface of the media. Thumbprints were made over the entire media surface, using as much of the thumbprint area as possible. For testing various surfaces, which included inanimate objects, various floors and other surfaces, sterile cotton swabs were dipped in clean bottled water, then the surfaces to be tested were streaked using the wet swab, being sure to turn the swab several times to expose all surfaces. The lid of a media plate was carefully removed and the swab used to inoculate the media on the plate by streaking in a zig-zag pattern, turning the plate 45 degrees, streaking again, and repeating the procedure once more, being sure that as much of the plate was streaked as possible using all the surface of the swab. After inoculation, the plates were labeled, inverted, and stored at room temperature (20-23° C.) for 48 to 96 hours. After inoculation, photos were taken using ambient light (sunlight) with lids removed to allow complete visualization of colonies and other microbial growth. At all times, aseptic techniques were used to minimize contamination. However, opening lids for photos at 48 hours could introduce contamination if plates were allowed to further grow for 96 hours. Those rare occasions are noted below. Application of Compel™, hand sanitizer, or other agents was done according to manufacturer instructions, where all surfaces were allowed to dry fully prior to re-testing.

### Example 4

### Test 1:3-Hour Hands, Computer Keyboard, and Office Chair.

First, the effectiveness of handwashing with soap and water versus the Compel™ formulation made in accordance with Example 2 was tested. Herein, the left thumb was tested prior to washing, and then immediately after dry. Second, the right thumb was tested prior to use of Compel™, then immediately after dry, then again 3 hours later following computer work and various activities at home. Finally, a leather office chair (arm rest) and a keyboard (spacebar) were tested prior to treatment, then again 3 hours after Compel™ and frequent use during that time. All plates recorded at 48 hours with no opening of plates prior to photos.

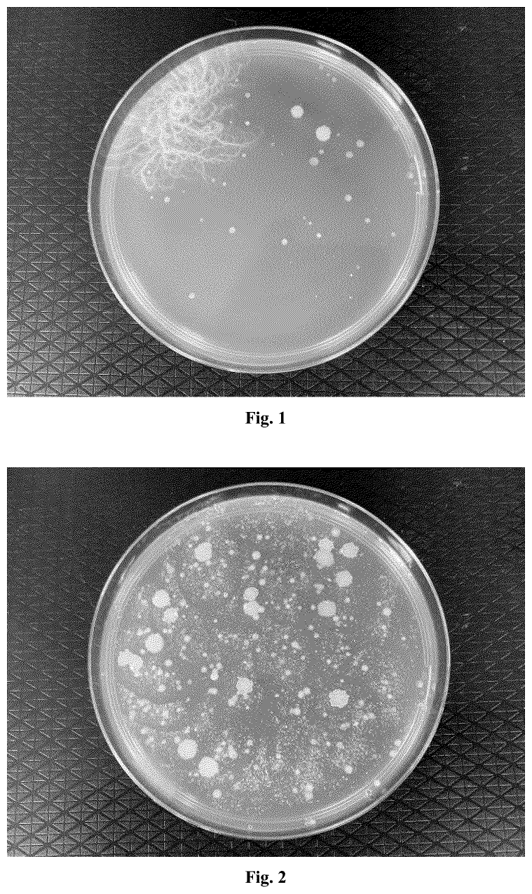

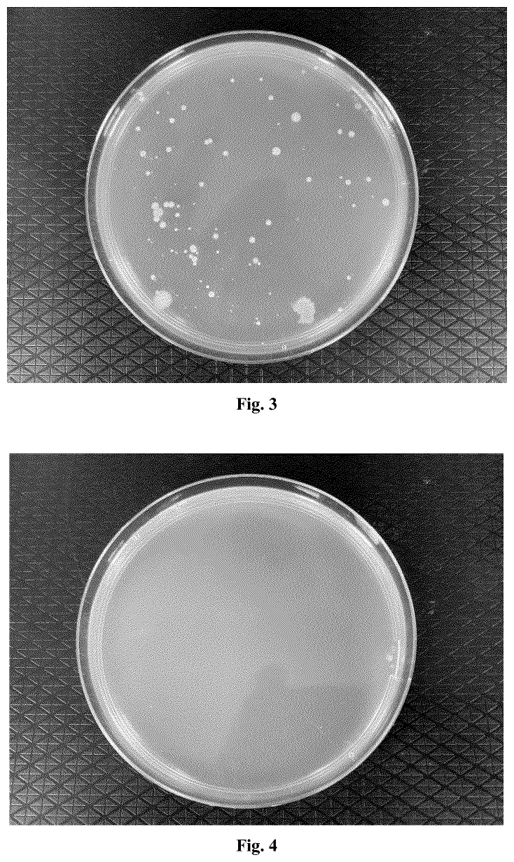

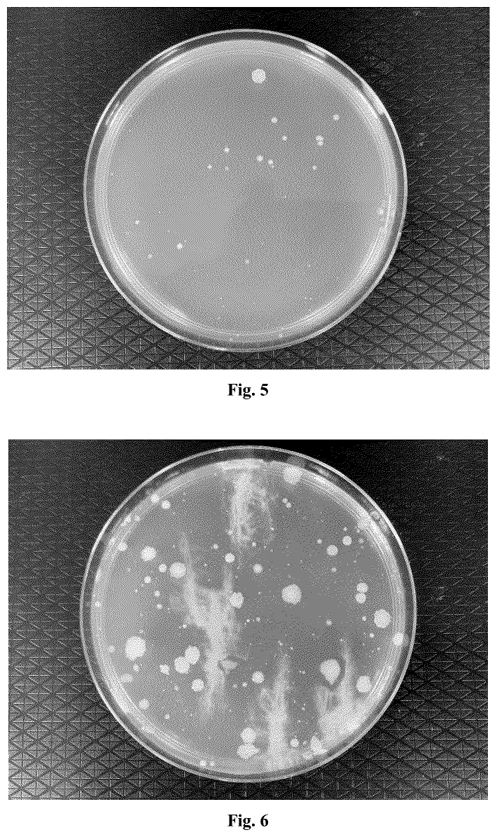

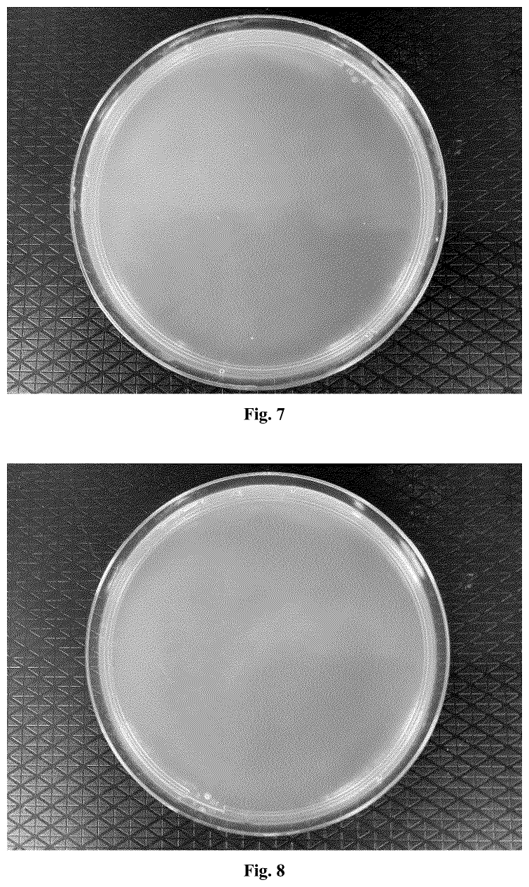

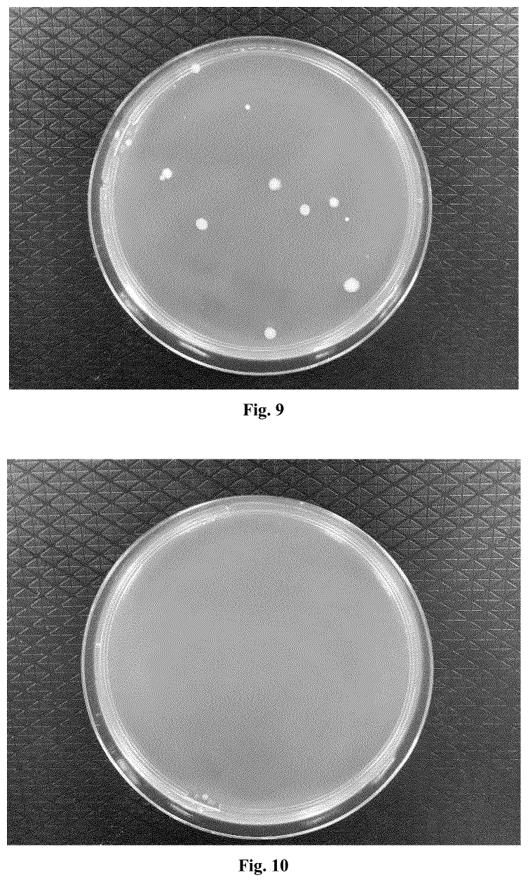

Results are shown in FIGS. 1-10, in which FIG. 1 depicts microorganism growth for “pre-wash LEFT thumb”; FIG. 2 depicts microorganism growth for “post-wash LEFT thumb”; FIG. 3 depicts microorganism growth for “pre-wash RIGHT thumb”; FIG. 4 depicts microorganism growth for “immediately after Compel™ RIGHT thumb”; FIG. 5 depicts microorganism growth for “3 hours after Compel™ RIGHT thumb”; FIG. 6 depicts microorganism growth for “keyboard prior to treatment”; FIG. 7 depicts microorganism growth for “keyboard immediately after Compel™”; FIG. 8 depicts microorganism growth for “keyboard 3 hours after Compel™”; FIG. 9 depicts microorganism growth for “office chair prior to treatment”; and FIG. 10 depicts microorganism growth for “office chair 3 hours after Compel™”.

### Test 1 Conclusions.

Use of soap and water to wash hands (left thumb) did not reduce microbial growth on test plates. In fact, often simple washing removes dirt and oils that would otherwise interfere with microbes sticking to the media. Thus, it is not a surprise to see more microorganism growth (number of colonies) after washing. However, these microbes are likely commensals (normal flora) and not pathogens from the environment.

Use of Compel™ on thumb (right) resulted in complete ablation of growth (no colonies evident) after immediate treatment. This is expected with an alcohol-based cleanser of sufficient strength (>60%). However, after 3 hours of computer-based work, regular household chores and other home-based activities, the number of colonies (each potentially representing one potential pathogen) was reduced. While a post wash test on this thumb was not performed, a comparison to the photo from the left thumb suggests a durability benefit of use of Compel™.

Use of Compel™ on the office chair (arm rest) and keyboard (spacebar) showed high durability/long-lasting protection in these tests. Herein, pre-treatment showed a wide range of growth. However, no growth was evident immediately after treatment (tested keyboard only), and this lack/prevention of growth persisted for up to the 3 hours (i.e. no colonies evident on Compel™-treated office chair or keyboard for up to three hours).

### Example 5

### Test 2: 24-Hour Refrigerator and PlayStation 4 Controller Test, Plus 4-Hour Thumb Testing.

First, effectiveness of Compel™ made in accordance with Example 2 was tested on two common surfaces, a stainless-steel refrigerator handle commonly used in a household of six persons, and a plastic PlayStation 4 (PS4) controller used very often throughout the day by two teenagers. Tests were done on these surfaces prior to treatment, as well as 8, 12, and 24 hours after use of Compel™. Second, to expand on understanding of growth as observed on thumb at 3 hours in test 1, we compared two thumbs, both treated with Compel™, but one left idle (LEFT thumb, no touching any surface or skin) for 4 hours, while the other (RIGHT thumb, touch) did routine tasks, including handling a TV remote, household tasks (no washing), touching skin, working on computer, and eating food over 4 hours. All plates were recorded at 48 hours with no opening of plates prior to photos.

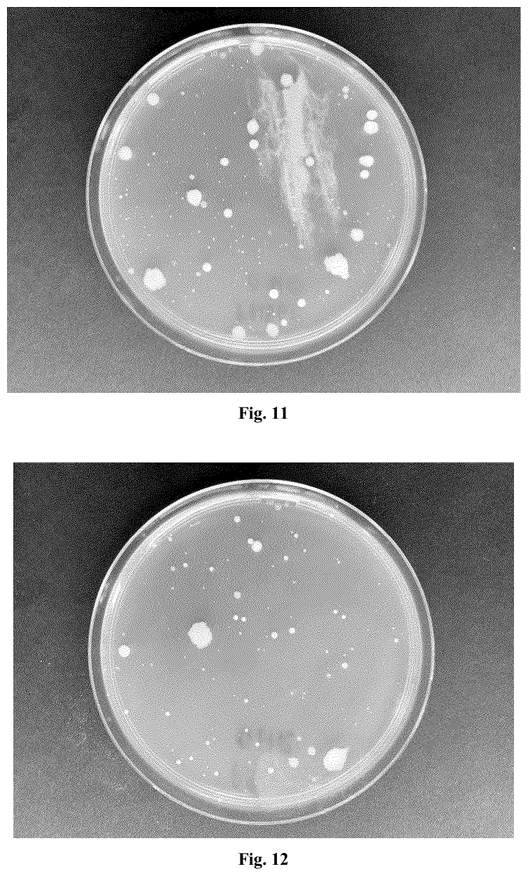

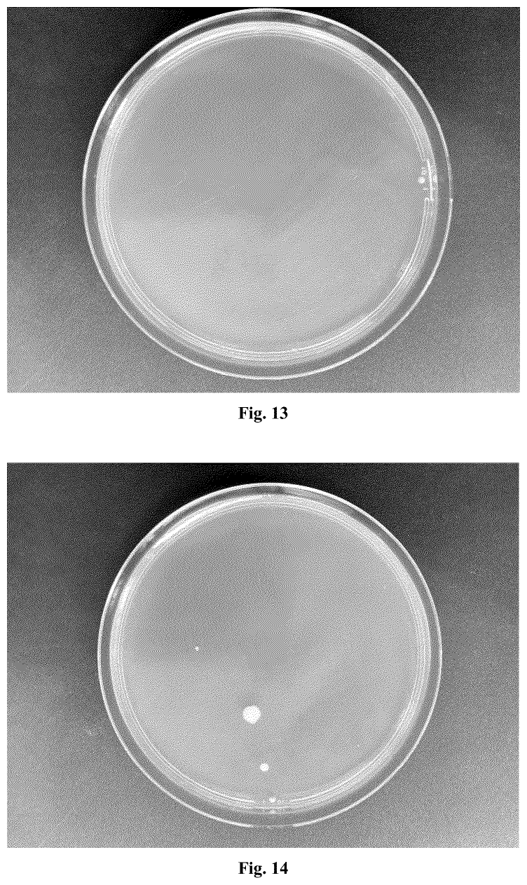

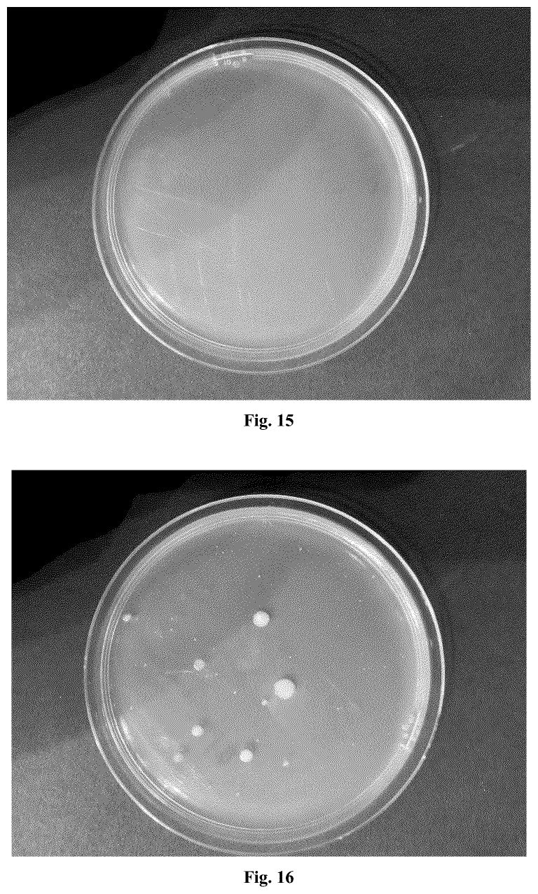

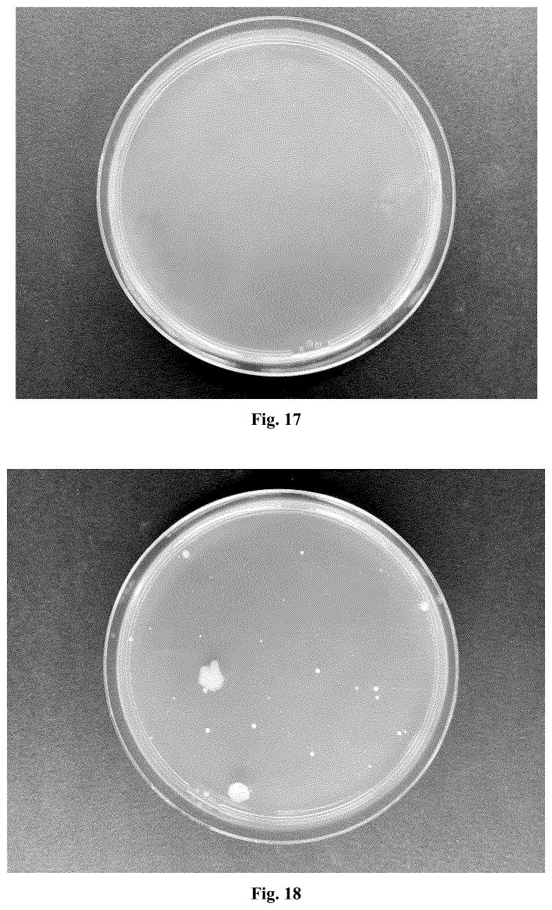

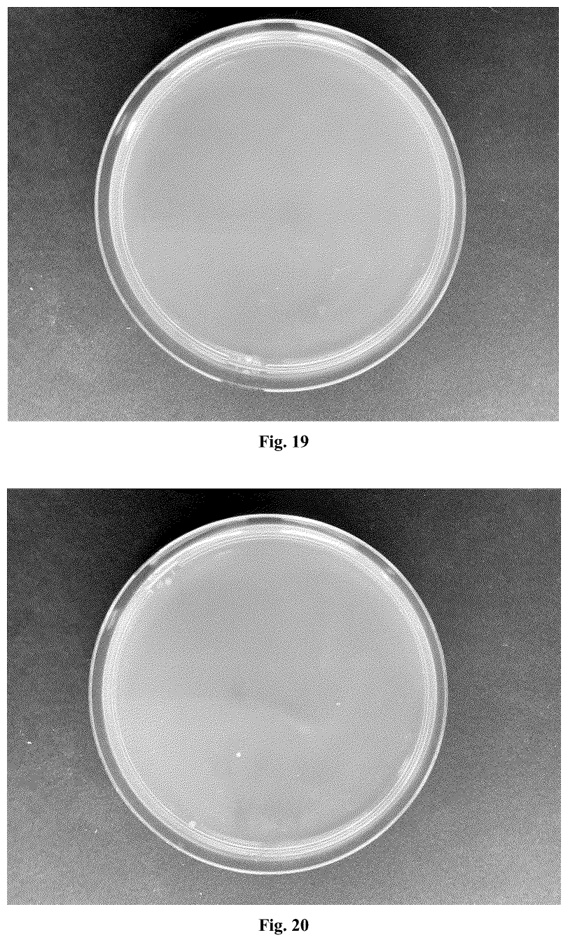

Results are shown in FIGS. 11-20, in which FIG. 11 depicts microorganism growth for “refrigerator handle before treatment”; FIG. 12 depicts microorganism growth for “PS4 controller before treatment”; FIG. 13 depicts microorganism growth for “refrigerator handle—8 hours post Compel™”; FIG. 14 depicts microorganism growth for “PS4 controller—8 hours post Compel™”; FIG. 15 depicts microorganism growth for “refrigerator handle—12 hours post Compel™”; FIG. 16 depicts microorganism growth for “PS4 controller—12 hours post Compel™”; FIG. 17 depicts microorganism growth for “refrigerator handle—24 hours post Compel™”; FIG. 18 depicts microorganism growth for “PS4 controller—24 hours post Compel™”; FIG. 19 depicts microorganism growth for “left thumb—4 hours post Compel™—no touch”; and FIG. 20 depicts microorganism growth for “right thumb-4 hours post Compel™-touch”.

### Test 2 Conclusions.

Use of Compel™ on refrigerator handle led to no visible growth at 8, 12, or even 24 hours. This is in stark contrast to the pre-treated surface which showed prolific growth, likely both bacterial and fungal. This handle surface was touched by numerous individuals throughout the day. The lack of growth post Compel™ suggests long-lasting/durable protection from contamination on this type of surface.

Use of Compel™ on PS4 controller led to a substantial reduction in colonies/growth at 8 hours, with reduced effectiveness at 12 and even less at 24 hours. This is not that surprising as this surface is constantly rubbed (handle of controller was swabbed), leading to potential removal of Compel™ via sweat and mechanical abrasion. Regardless, all time-points, including 24 hours, were clearly improved compared to the pre-treated test plate.

Use of Compel™ on skin was tested by applying it on both thumbs and then comparing the untouched thumb to the one doing normal tasks over 4 hours. No pre-treatment was done, as this had been done previously (see test 1 for examples). Impressively, the left thumb (no touch) showed no growth, while the right thumb (touch) showed minimal growth. It is likely the growth on the right thumb occurred due to environmental contact and/or skin or mucous membranes (eating). Nevertheless, this small amount of growth adds to the observations from test 1 suggesting durability/long-lasting protection with use of Compel™ even on skin.

### Example 6

### Test 3—Part A: Effectiveness Testing of Compel™ on School Wrestling Mats in Active Use.

First, a test section of the wrestling mat labeled “area A” was sampled prior to treatment (Note: area A was previously cleaned with standard cleaners after previous wrestling team practice but not cleaned again prior to current practice). The protocol normally used at the subject school for wrestling mat cleaning is as follows: after practices, the team cleans the entire mat surface with MadaCide-1, a commercially-available disinfectant cleaner (Mada Medical Products, Inc., Carlstadt, New Jersey). For comparison, we tested another section of the mat (labeled “area B”) after cleaning with MadaCide and fully drying. In addition, area A was cleaned with Compel™ made in accordance with Example 1 prior to practice, and tests taken immediately after treatment, as well as 1.5 hours later (after full wresting team practice using this area of mat). Second, for comparison of durability with repeated practices, a test was performed on another area of the wrestling mat (“area C”) that had been treated with the Compel™ a few days earlier. All plates recorded at 48 hours with no opening of plates prior to photos.

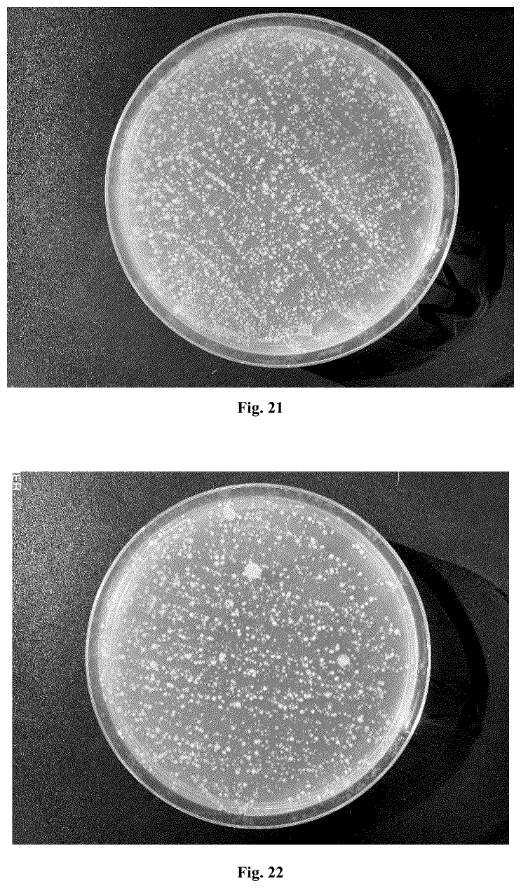

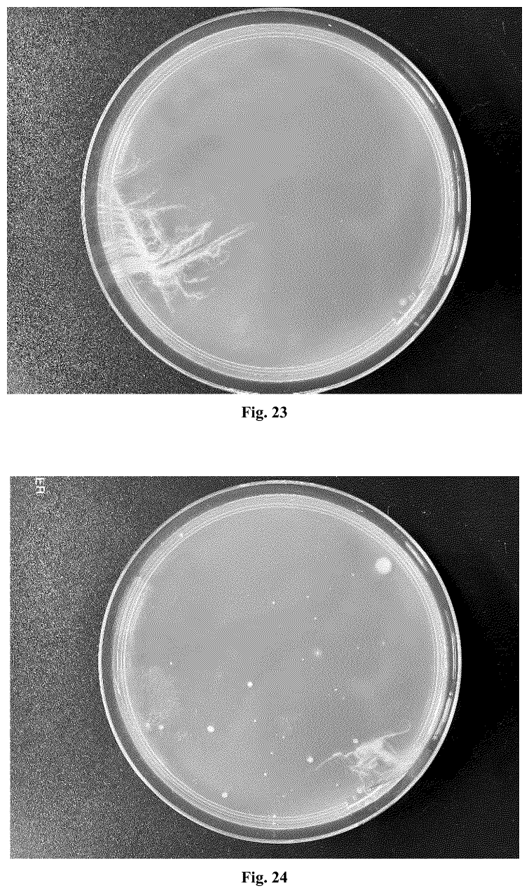

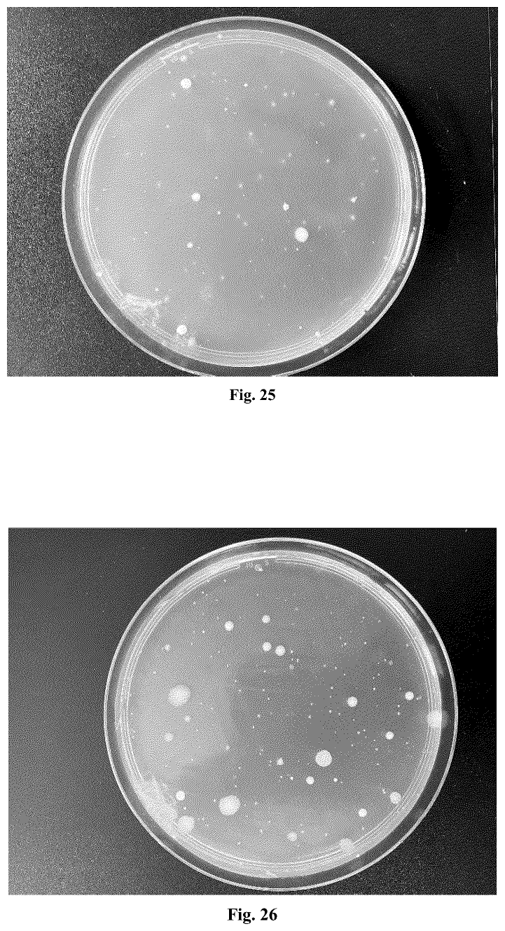

Results are shown in FIGS. 21-25, in which FIG. 21 depicts microorganism growth for “wrestling mat area A prior to treatment”; FIG. 22 depicts microorganism growth for “wrestling mat area B immediately after MadaCide”; FIG. 23 depicts microorganism growth for “wrestling mat area A immediately after Compel™ treatment”; FIG. 24 depicts microorganism growth for “wrestling mat area A 1.5 hours after Compel™ treatment”; and FIG. 25 depicts microorganism growth for “wrestling mat area C 2 days after Compel™ treatment”.

### Test 3—Part A Conclusions.

Lower room wrestling mat area A showed abundant growth prior to treatment. This is not surprising given the number of practices a week (up to 10) and number of wrestlers (100+). However, this mat is routinely cleaned after each practice by large mops and use of MadaCide cleaner. Of particular interest is the test performed immediately after MadaCide was used to clean a separate part of the mat (area B) in the lower room. Herein, the amount of growth was very similar to that on area A prior to treatment with Compel™. This suggests that either the product or the application was insufficient to disinfect this area. This is in stark contrast to substantial reductions in growth observed immediately after application of Compel™ or 1.5 hours later after wrestling practice was completed. In fact, another area of the lower room mat (area C) was treated with Compel™ several days earlier. When it was tested, growth was observed, but dramatically less than observed in non-Compel™ treated areas (area A prior to treatment or area B).

### Example 7

### Test 3—Part B: Effectiveness of Compel™ on Wrestlers' Hands Before and After Practice.

Tests were conducted on wrestling partners, Wrestler A (before and after Compel™ made in accordance with Example 1) and Wrestler B (before and after hand sanitizer-active ingredient 62% ethyl alcohol). In this case, water moistened sterile cotton swabs were used to test subjects' hands, specifically palms, as opposed to just thumbs. All plates recorded at 48 hours with no opening of plates prior to photos.

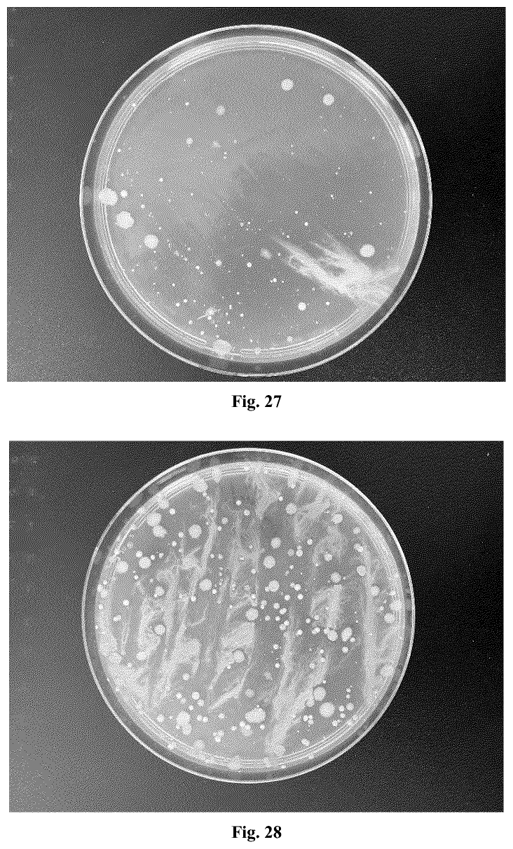

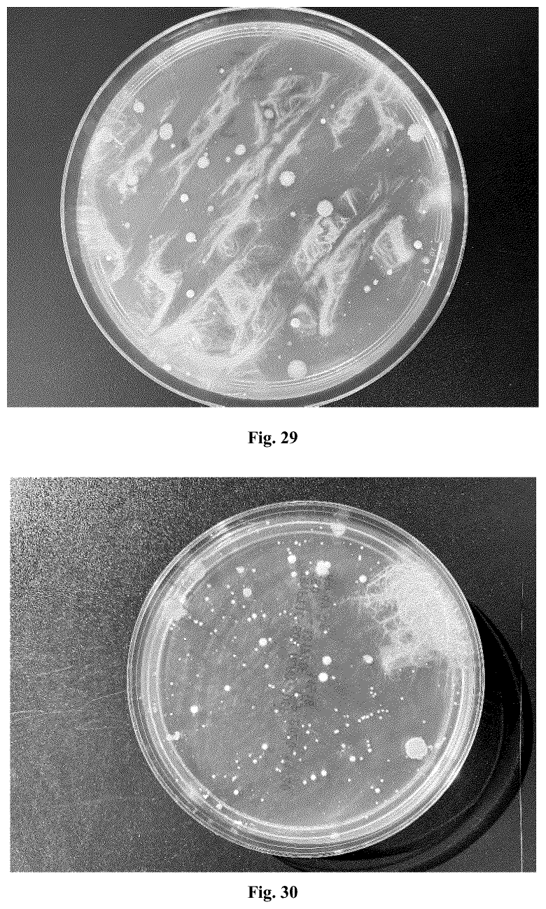

Results are shown in FIGS. 26-29, in which FIG. 26 depicts microorganism growth for “wrestler A prior to treatment”; FIG. 27 depicts microorganism growth for “wrestler A post Compel™ and full practice”; FIG. 28 depicts microorganism growth for “wrestler B prior to treatment”; and FIG. 29 depicts microorganism growth for “wrestler B post hand sanitizer and full practice”.

### Test 3—Part B Conclusions.

Both wrestlers showed growth both before and after wrestling practice when treated with Compel™ or hand sanitizer. It is hard to compare effectiveness between treatments, as both wrestlers had different pre-treatment levels. This test appears inconclusive, especially since Wrestler A may have used Compel™ previously.

### Example 8

### Test 3—Part C: Effectiveness of Compel™ on Wrestling Mats and Wrestling Scale.

First, tests were conducted on two different wrestling rooms. Specifically, wrestling mat on upper level was tested prior to treatment with Compel™, immediately after use of Compel™ made in accordance with Example 1, and 1 week after treatment with Compel™. In addition, mat on lower level (area A on mat as tested previously) was tested at 24 hours and again at 1 week after treatment with Compel™. Second, the wrestling scale touch pad was tested prior to, and then immediately after Compel™, while the foot pad was tested prior to treatment (no follow-up tests). All plates recorded at 48 hours with no opening of plates prior to photos.

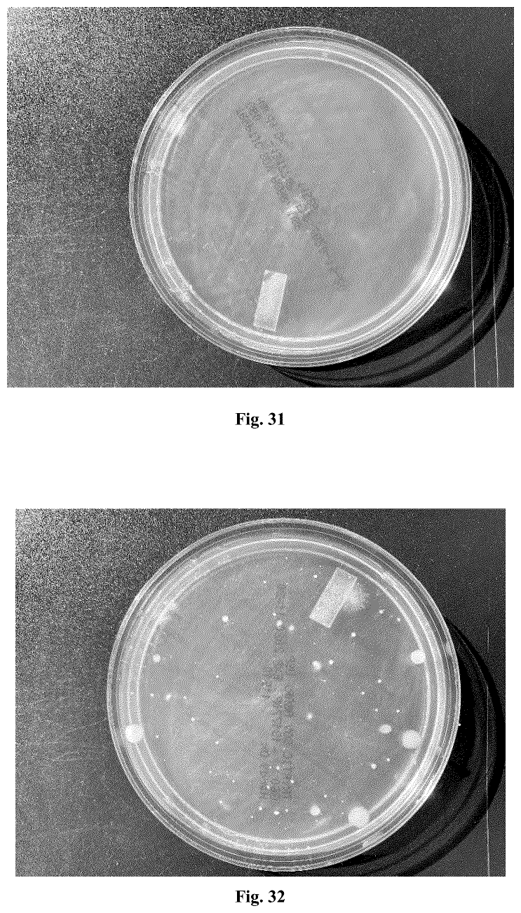

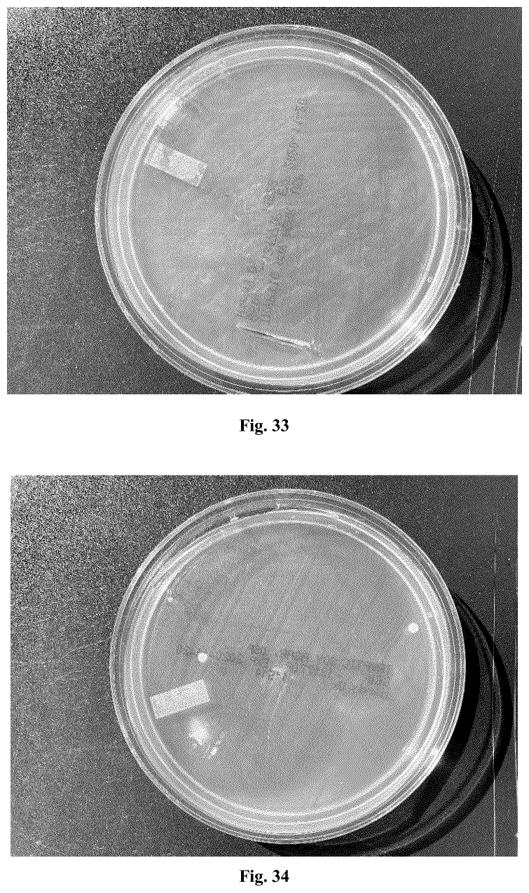

Results are shown in FIGS. 30-37, in which FIG. 30 depicts microorganism growth for “upper wrestling room mat prior to treatment”; FIG. 31 depicts microorganism growth for “upper wresting room mat immediately after Compel™ treatment”; FIG. 32 depicts microorganism growth for “wrestling scale touch pad prior to treatment”; FIG. 33 depicts microorganism growth for “wrestling scale touch pad immediately after Compel™ treatment”;

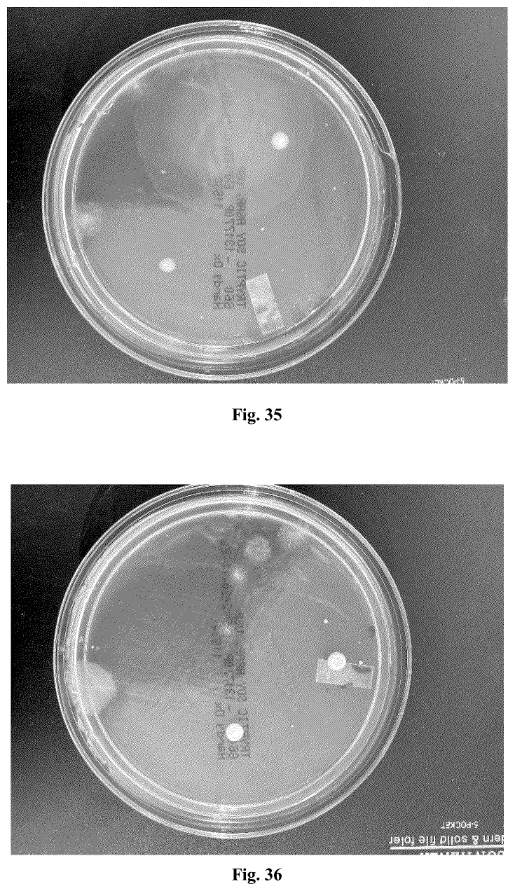

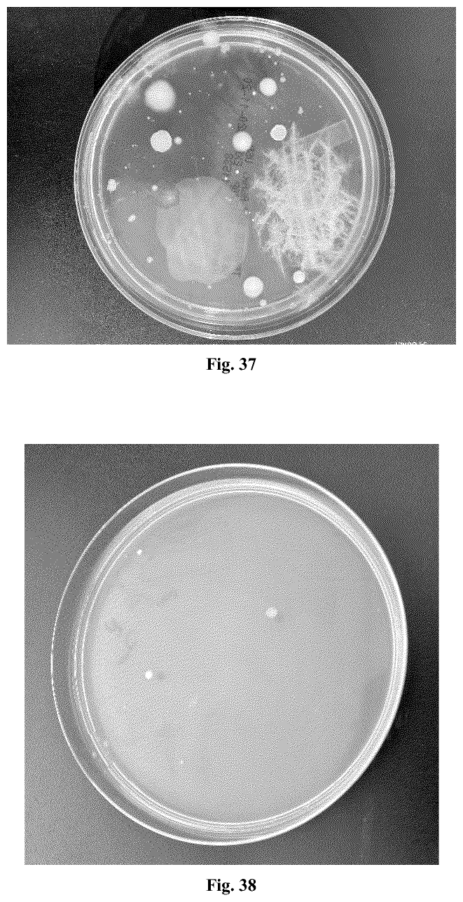

FIG. 34 depicts microorganism growth for “lower room mat area A at 24 hours post Compel™ treatment”; FIG. 35 depicts microorganism growth for “lower room mat area A at 1 week post Compel™ treatment”; FIG. 36 depicts microorganism growth for “upper room mat 1 week post Compel™ treatment”; and FIG. 37 depicts microorganism growth for “wrestling scale foot pad pre-treatment”.

### Test 3—Part C Conclusions.

Upper room—Tests were performed on wrestling mat prior to practice and prior to treatment with Compel™, then again immediately after treatment, and again a week later (after repeated use). As expected, prior to treatment, there was copious growth of microbes (likely bacterial and fungal) on the wrestling mats. However, immediately after Compel™, no colonies or other growth was evident. A week later, levels of microbes were dramatically reduced compared to pre-treatment levels.
Lower room—Test on wrestling mat area A at 24 hours post treatment with Compel™ occurred after 2 practices and a water wash (wet mop pads with water). Impressively, only minimal growth was observed at 24 hours, with only a handful of colonies present even 1 week after treatment.
Scale—The touch pad on the scale is routinely handled (e.g. daily) to turn on and off the machine, while the foot pad is touched by many individuals, often without socks. As expected, tests on both surfaces led to abundant growth, with the worst observed on the foot pad. Only one post Compel™ test was performed, specifically on the touch pad. As expected, no growth was observed.

### Example 9

### Test 4: Places of Worship.

This series of tests focused on two churches (Church A in Franklin, IN and Church B in Mooresville, IN). Both churches were tested after all Sunday services (Sunday afternoon, day 1) for pre-treatment, immediately after Compel™ made in accordance with Example 1 was applied (day 1), and then again one week later on Sunday afternoon (day 8). Testing was performed on 5 common use areas per church, including restroom entrances, public countertops, children's area check-in, handrail to stairs, and exit door handles. Tests included pre-treatment, immediate post Compel™, and 1-week post Compel™, so each church used 10 plates on day 1 and 5 plates on day 8. Many of these pre-treatment tests (day 1) were performed on surfaces routinely cleaned with other agents, so a huge amount of growth was not expected. Pre-treatment tests and immediately post treatment with Compel™ plates (from day 1) were photographed at 48 hours by opening the plates. However, the sizes of the colonies were small. So, plates were allowed an additional 2 days of growth to increase size. Nevertheless, it should be mentioned that opening of plates at 48 hours may have led to some contamination on these plates, although care was taken to prevent contamination. For tests performed a week later (day 8), we allowed the full 96 hours of growth (no opening of plates until photos were taken at 96 hours). All photos (48 and 96 hours) will be included here for full disclosure.

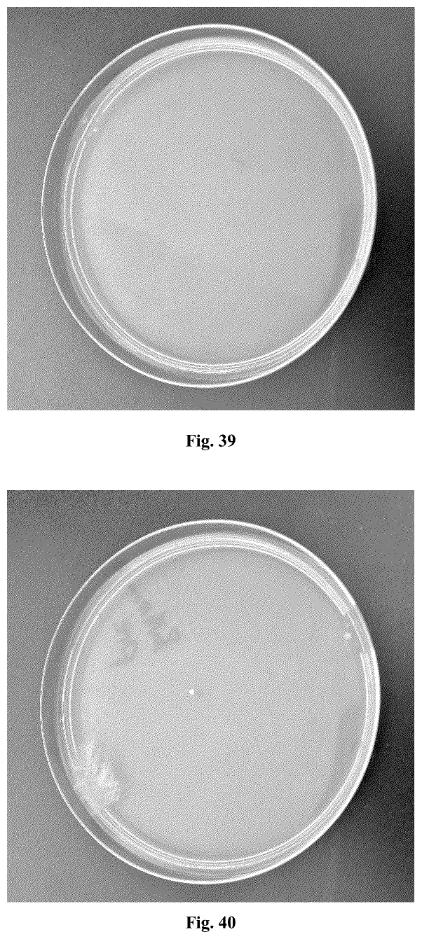

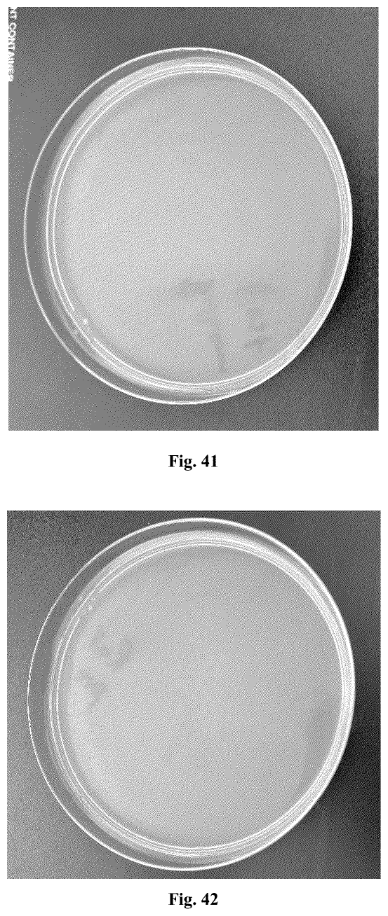

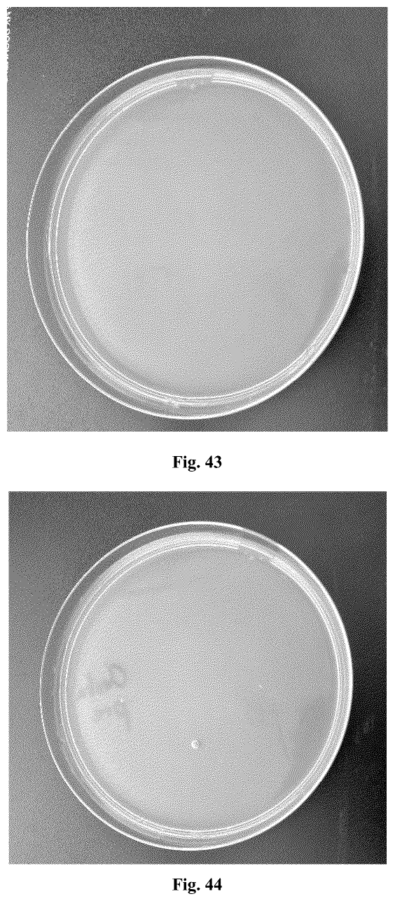

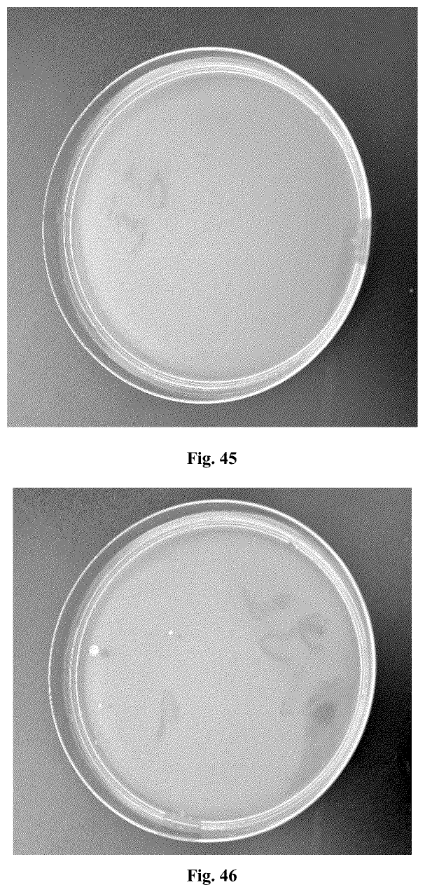

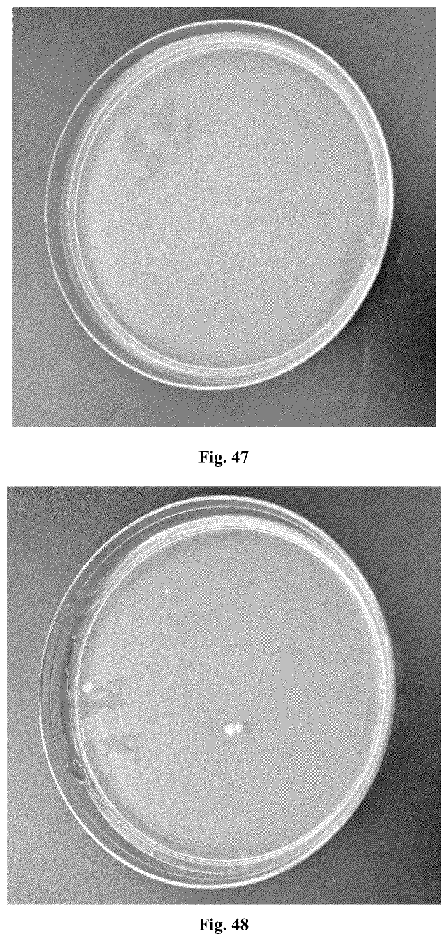

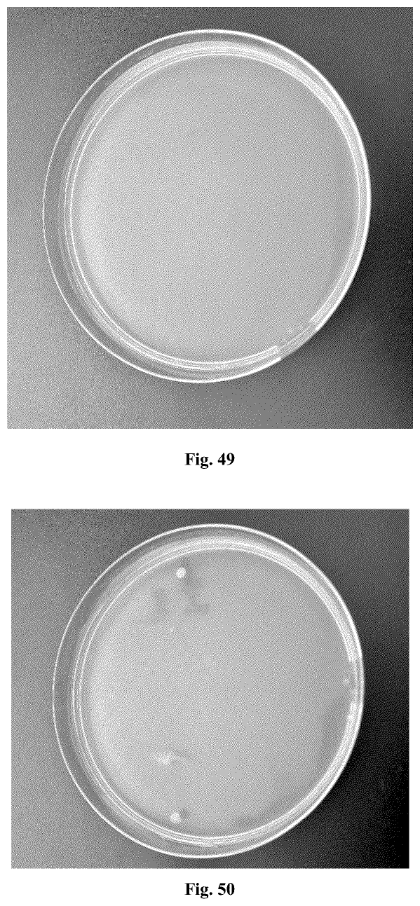

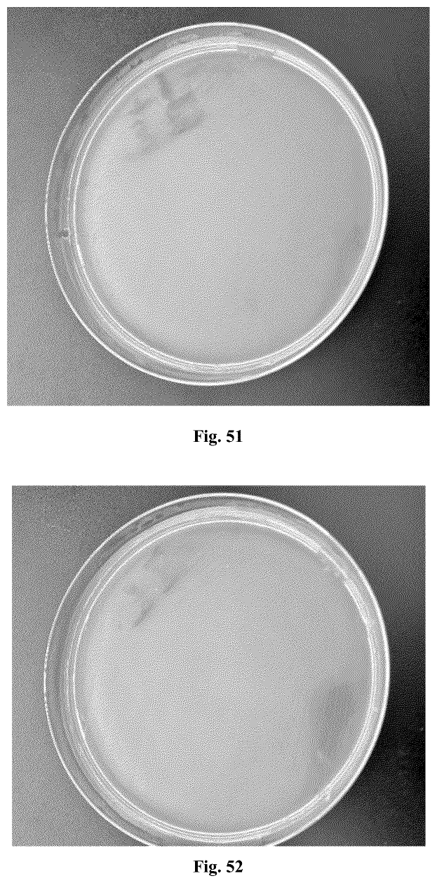

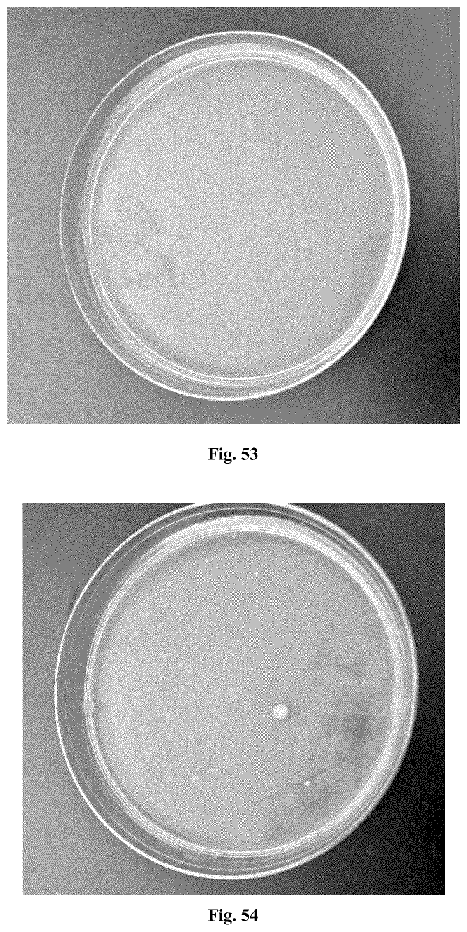

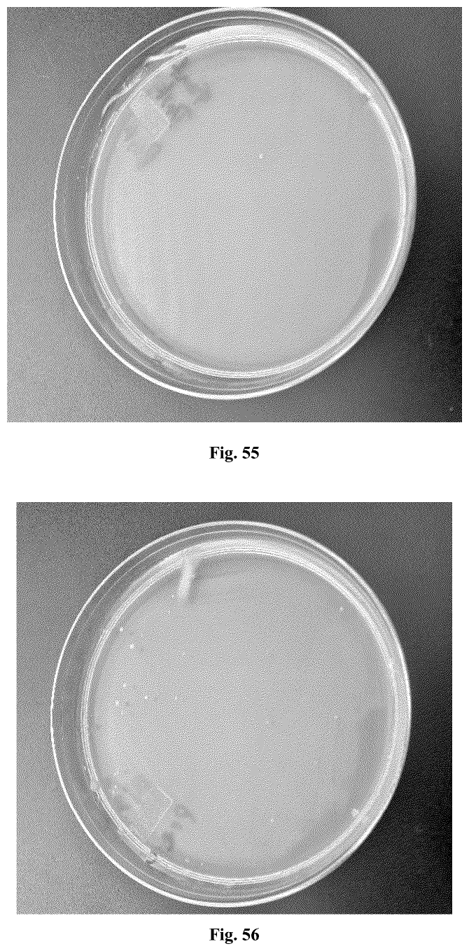

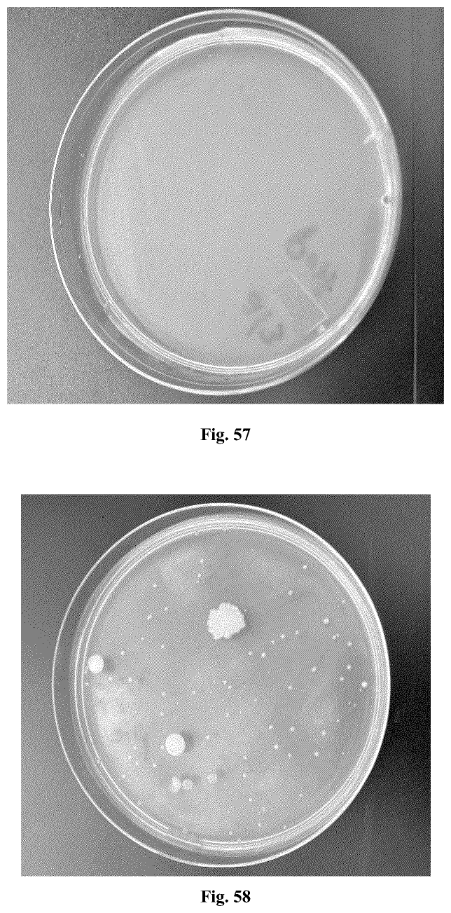

Results of Test 4—Part A, 48 HOUR GROWTH OF PRE-TREATMENT AND IMMEDIATE COMPEL™ TREATMENT, are depicted in FIGS. 38-57, in which FIG. 38 depicts microorganism growth for “pre-treatment—Church B Front door (48 hrs)”; FIG. 39 depicts microorganism growth for “immediately post Compel™—Church B Front door (48 hrs)”; FIG. 40 depicts microorganism growth for “pre-treatment—Church B Bathroom door (48 hrs)”; FIG. 41 depicts microorganism growth for “immediately post Compel™—Church B Bathroom door (48 hrs)”; FIG. 42 depicts microorganism growth for “pre-treatment—Church B Hand rail (48 hrs)”; FIG. 43 depicts microorganism growth for “immediately post Compel™—Church B Hand rail (48 hrs)”; FIG. 44 depicts microorganism growth for “pre-treatment—Church B Check-in (48 hrs)”; FIG. 45 depicts microorganism growth for “immediately post Compel™—Church B Check-in (48 hrs)”; FIG. 46 depicts microorganism growth for “pre-treatment-Church B Café (48 hrs)”; FIG. 47 depicts microorganism growth for “immediately post Compel™—Church B Café (48 hrs)”; FIG. 48 depicts microorganism growth for “pre-treatment—Church A Front door (48 hrs)”; FIG. 49 depicts microorganism growth for “immediately post Compel™—Church A Front door (48 hrs)”; FIG. 50 depicts microorganism growth for “pre-treatment—Church A Bathroom entry (48 hrs)”; FIG. 51 depicts microorganism growth for “immediately post Compel™—Church A Bathroom door (48 hrs)”; FIG. 52 depicts microorganism growth for “pre-treatment—Church A Hand rail (48 hrs)”; FIG. 53 depicts microorganism growth for “immediately post Compel™—Church A Hand rail (48 hrs)”; FIG. 54 depicts microorganism growth for “pre-treatment—Church A Front desk (48 hrs)”; FIG. 55 depicts microorganism growth for “immediately post Compel™—Church A Front desk (48 hrs)”; FIG. 56 depicts microorganism growth for “pre-treatment—Church A 2s and 3s table (48 hrs)”; and FIG. 57 depicts microorganism growth for “immediately post Compel™—Church A 2s and 3s table (48 hrs)”.

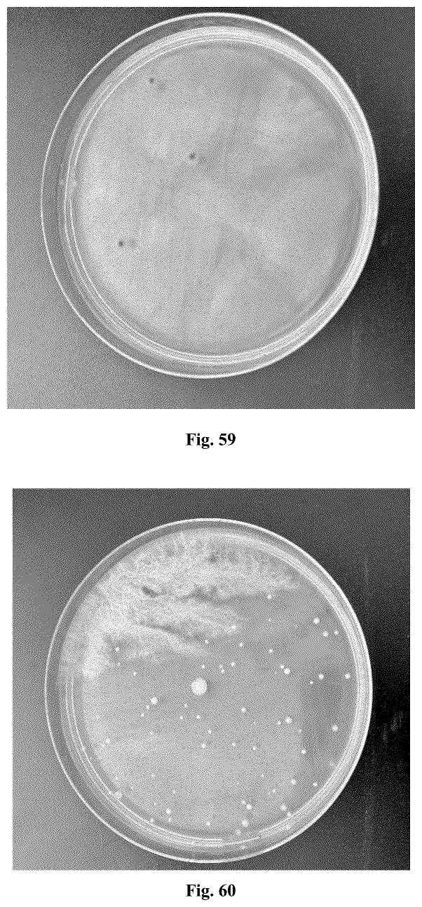

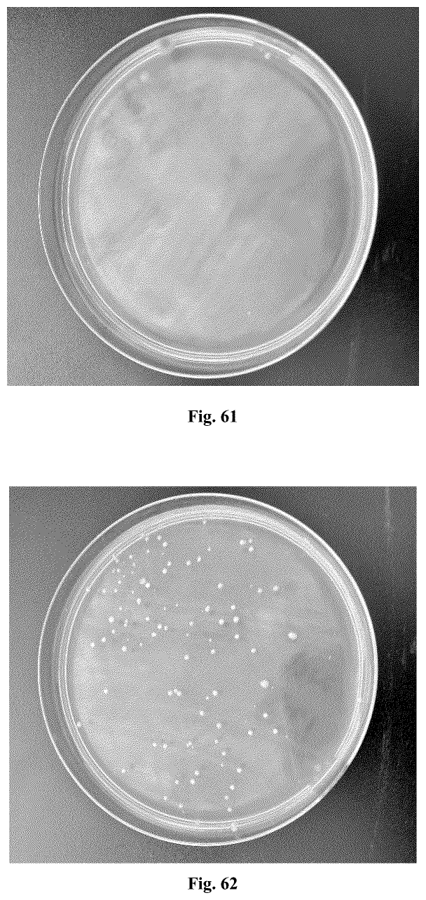

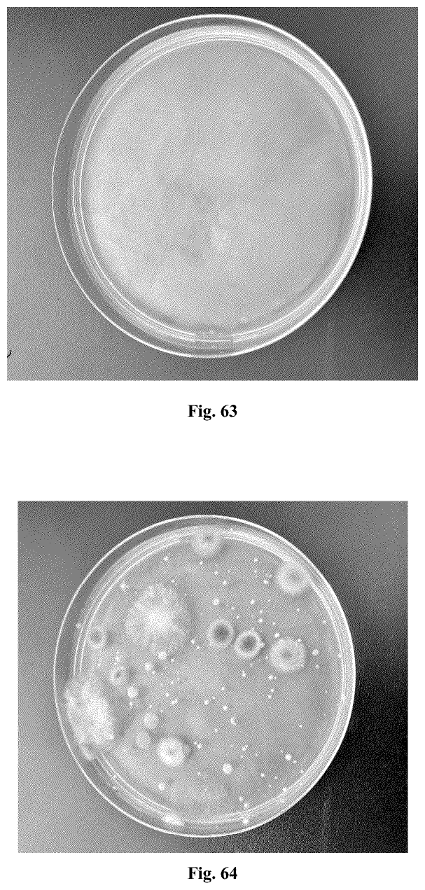

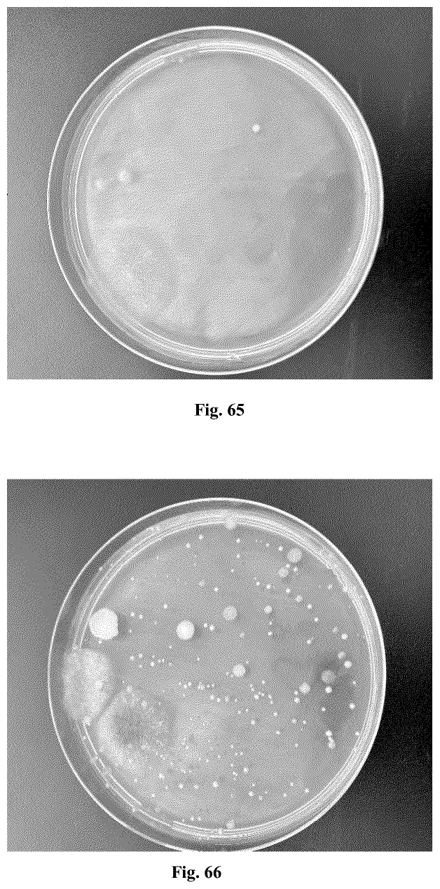

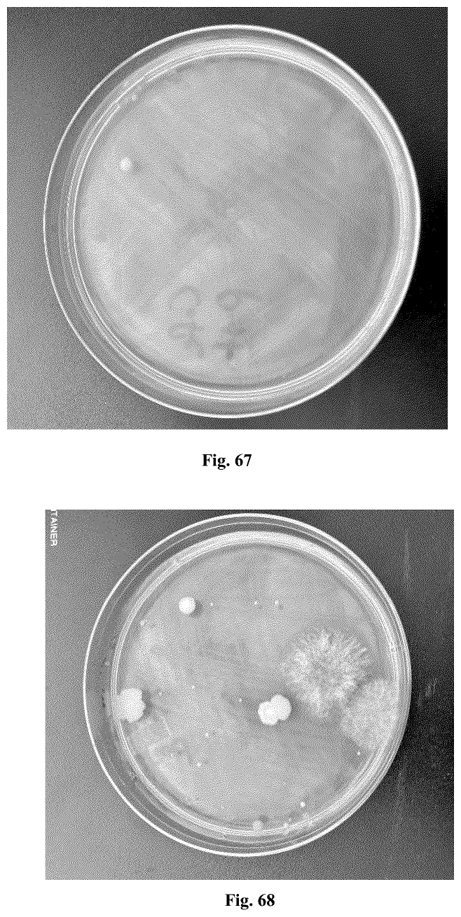

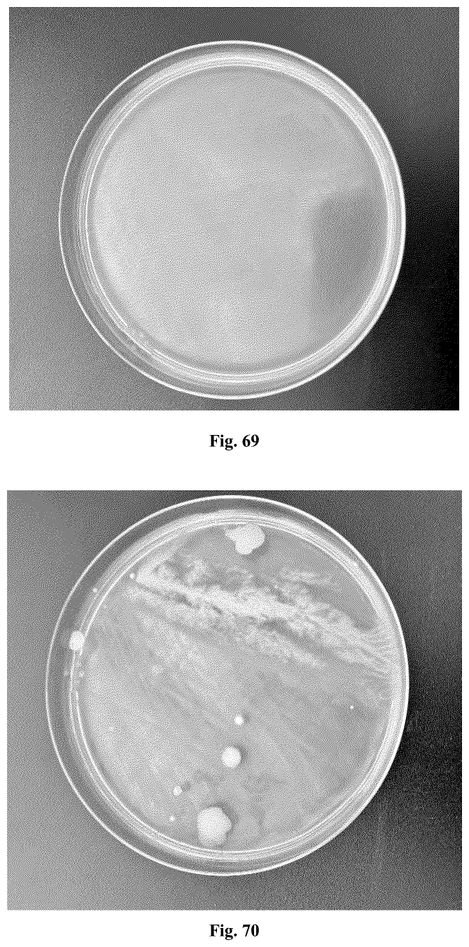

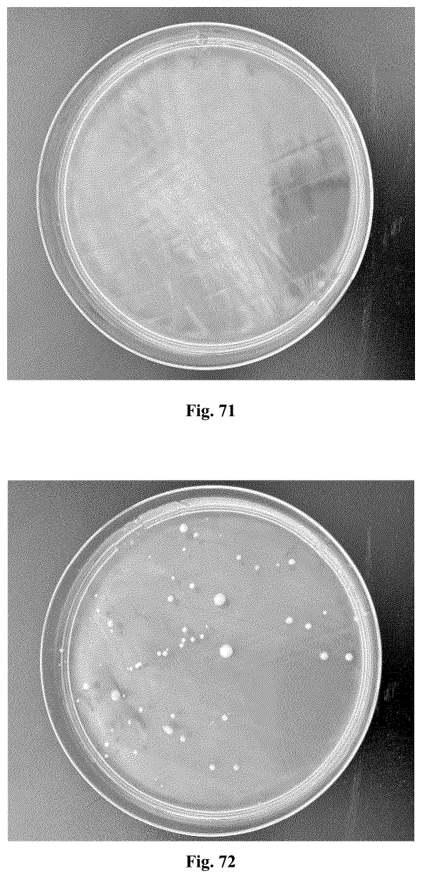

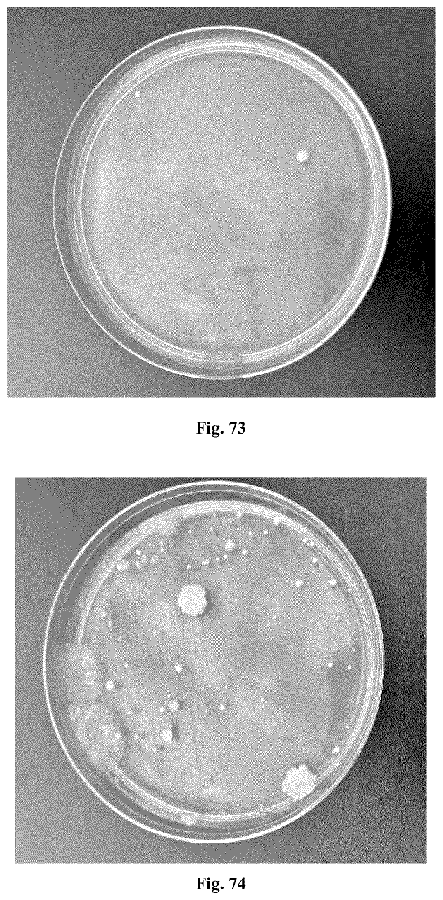

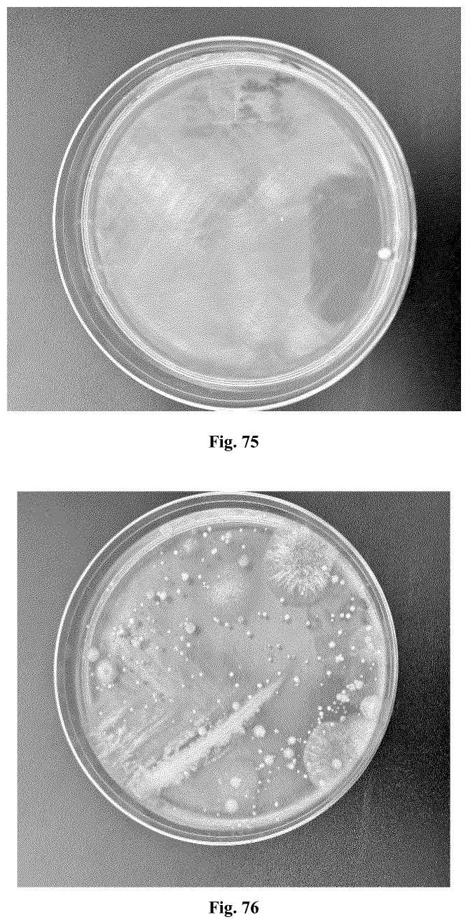

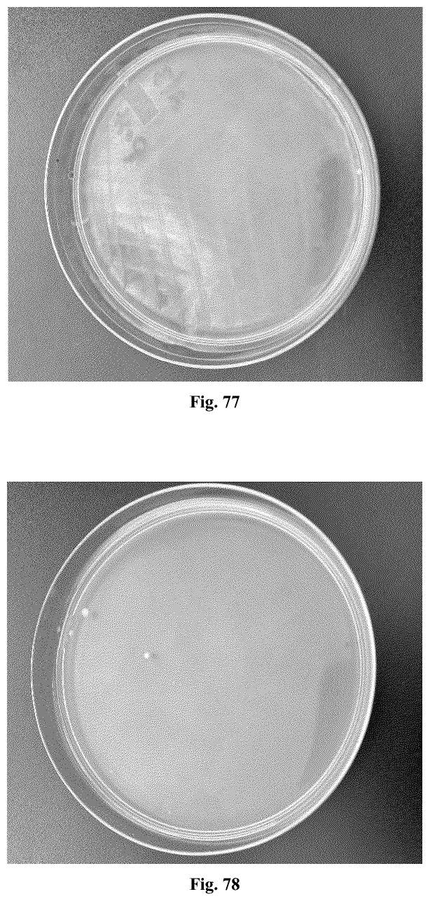

Results of Test 4—Part B, 96 HOUR GROWTH OF PRE-TREATMENT AND IMMEDIATE COMPEL™ TREATMENT, are depicted in FIGS. 58-77, in which FIG. 58 depicts microorganism growth for “pre-treatment—Church B Front door (96 hrs)”; FIG. 59 depicts microorganism growth for “immediately post Compel™—Church B Front door (96 hrs)”; FIG. 60 depicts microorganism growth for “pre-treatment—Church B Bathroom door (96 hrs)”; FIG. 61 depicts microorganism growth for “immediately post Compel™—Church B Bathroom door (96 hrs)”; FIG. 62 depicts microorganism growth for “pre-treatment—Church B Hand rail (96 hrs)”; FIG. 63 depicts microorganism growth for “immediately post Compel™—Church B Hand rail (96 hrs)”; FIG. 64 depicts microorganism growth for “pre-treatment—Church B Check-in (96 hrs)”; FIG. 65 depicts microorganism growth for “immediately post Compel™—Church B Check-in (96 hrs)”; FIG. 66 depicts microorganism growth for “pre-treatment-Church B Café (96 hrs)”; FIG. 67 depicts microorganism growth for “immediately post Compel™—Church B Café (96 hrs)”; FIG. 68 depicts microorganism growth for “pre-treatment—Church A Front door (96 hrs)”; FIG. 69 depicts microorganism growth for “immediately post Compel™—Church A Front door (96 hrs)”; FIG. 70 depicts microorganism growth for “Pre-treatment—Church A Bathroom entry (96 hrs)”; FIG. 71 depicts microorganism growth for “Immediately post Compel™—Church A Bathroom door (96 hrs)”; FIG. 72 depicts microorganism growth for “Pre-treatment—Church A Hand rail (96 hrs)”; FIG. 73 depicts microorganism growth for “Immediately post Compel™—Church A Hand rail (96 hrs)”; FIG. 74 depicts microorganism growth for “Pre-treatment—Church A Front desk (96 hrs)”; FIG. 75 depicts microorganism growth for “Immediately post Compel™—Church A Front desk (96 hrs)”; FIG. 76 depicts microorganism growth for “Pre-treatment—Church A 2s and 3s table (96 hrs)”; and FIG. 77 depicts microorganism growth for “Immediately post Compel™—Church A 2s and 3s table (96 hrs)”.

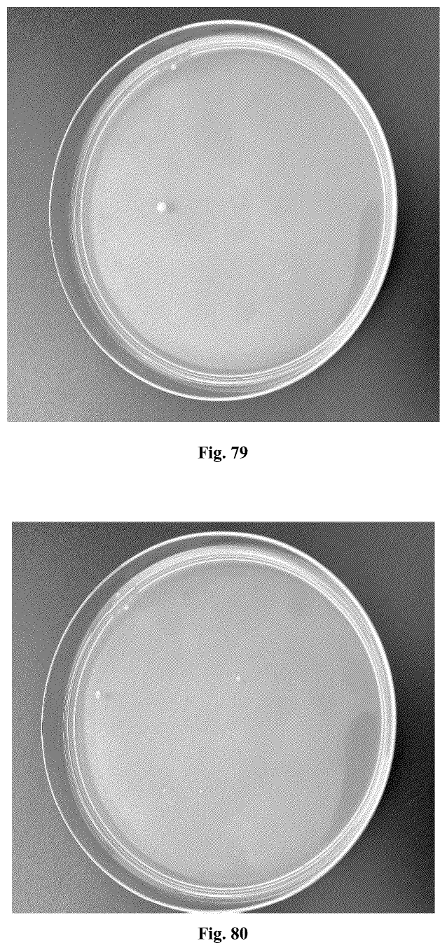

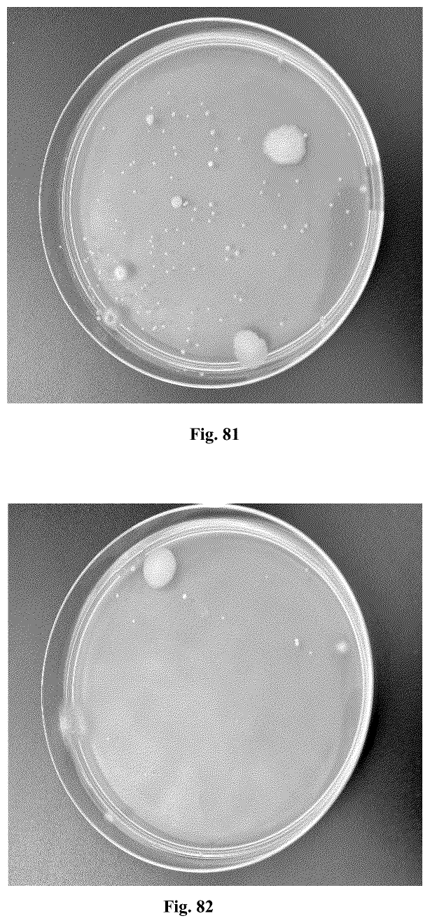

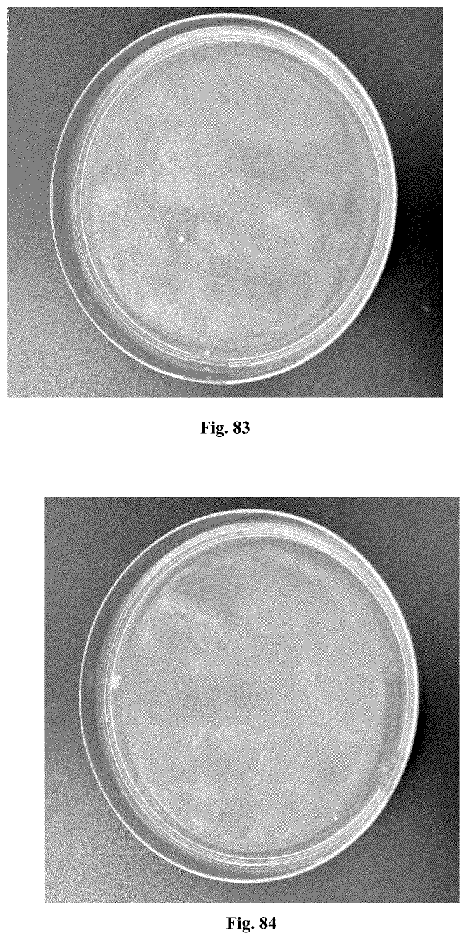

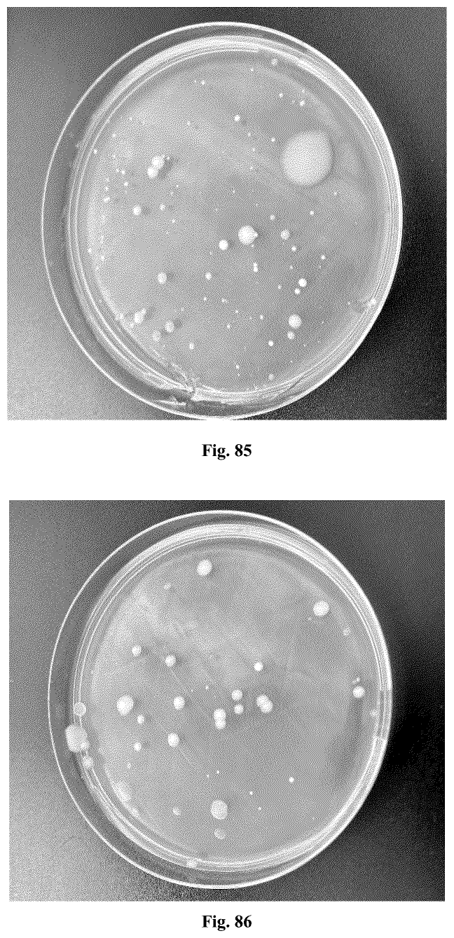

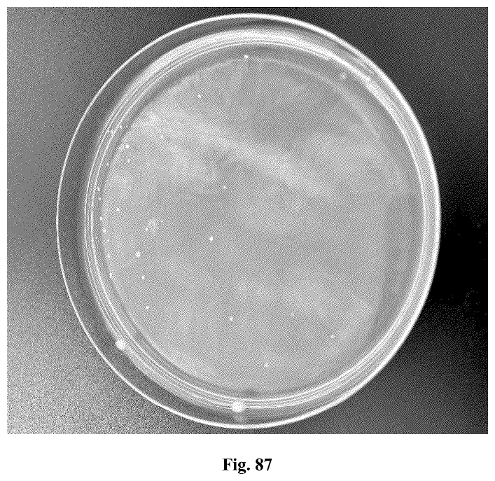

Results of Test 4—Part C, 1 WEEK POST COMPEL™ TREATMENT, are depicted in FIGS. 78-87, in which FIG. 78 depicts microorganism growth for “1 week post Compel™—Church B Front door (96 hrs)”; FIG. 79 depicts microorganism growth for “1 week post Compel™—Church B Bathroom door (96 hrs)”; FIG. 80 depicts microorganism growth for “1 week post Compel™—Church B Hand rail (96 hrs)”; FIG. 81 depicts microorganism growth for “1 week post Compel™—Church B Children area check-in (96 hrs)”; FIG. 82 depicts microorganism growth for “1 week post Compel™—Church B Café countertop (96 hrs)”; FIG. 83 depicts microorganism growth for “1 week post Compel™—Church A Front entry door (96 hrs)”; FIG. 84 depicts microorganism growth for “1 week post Compel™—Church A Bathroom door (96 hrs)”; FIG. 85 depicts microorganism growth for “1 week post Compel™—Church A Hand rail (96 hrs, broken plate)”; FIG. 86 depicts microorganism growth for “1 week post Compel™—Church A Front desk (96 hrs, check-in)”; and FIG. 87 depicts microorganism growth for “1 week post Compel™—Church A 2s and 3s tabletop (96 hrs)”.

### Test 4 Conclusions.

### Notes:

a. A few plates had an air bubble which can be confused with growth. This would not interfere with the test. These imperfections were caused when plates were prepared in a sterile environment.
b. Church A handrail plate at 1-week post treatment had broken lid and plate, likely leading to contamination. This was noted prior to testing. While we still used this plate for testing, these results are questionable, as it is unclear if contamination occurred after or even prior to the test.
c. Overall, 48-hour growth was not sufficient to produce large enough colonies to be easily visible. While it did not appear that any plate treated with Compel™ and then immediately tested had growth, it cannot be conclusively shown by these photos. Thus, these plates were allowed to grow for another two days, 96 hours total. The time exposed at 48 hours was minimal, only for a few seconds each to collect the photo.

Not surprisingly, surfaces routinely touched by numerous individuals (check-in, 2s and 3s table, etc.) showed the most growth on pre-treatment plates. Surprisingly, the café had abundant growth despite being recently cleaned by workers during service as well as custodial staff.

With 96-hours of growth, the immediate post treatment with Compel™ plates were quite clean, only showing a few colonies if any at all. It is not clear if these are contamination due to opening at 48 hours or were present on original surface. Nevertheless, Compel™ dramatically reduced growth on all surfaces.

On testing surfaces at a week post Compel™ treatment, the growth was dramatically reduced compared to pre-treatment plates, suggesting a more effective treatment/process than done prior to day 1 testing. While no plate was clean, growth on all surfaces was reduced with use of Compel™.

These tests demonstrate effective reductions in viable microorganisms on numerous surfaces with use of Compel™. While the magnitude of the reduction on skin is variable depending on various conditions, reductions on surfaces are very dramatic, often showing little to no growth, even after extended periods of time. Altogether, these data suggest a long-lasting or durable effect produced by Compel™ beyond what would be expected with soap and water or conventional alcohol-based hand sanitizers. While species identification was not explored, these results can suggest that Compel™ kills or prevents the growth of microorganisms on many surfaces, which could include pathogens.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A topical disinfectant composition for sanitizing a surface or increasing the surface's resistance to microbe transmission, the composition comprising:
ethyl alcohol;
chlorhexidine;
an ester-containing moisturizer; and
water.

2. The composition according to claim 1 wherein the ester-containing moisturizer comprises gluconolactone.

3. The composition according to claim 1 or claim 2, further comprising a surfactant.

4. The composition according to claim 3 wherein the surfactant comprises lauramine oxide.

5. The composition according to any of claims 1-4, further comprising a preservative.

6. The composition according to any of claims 1-5, further comprising colloidal silver.

7. The composition according to any of claims 1-6, further comprising hydrogen peroxide.

8. A method for sanitizing a surface or increasing the surface's resistance to microbe transmission, comprising:
providing a composition comprising ethyl alcohol, chlorhexidine, an ester-containing moisturizer, and water;
selecting a surface to be sanitized;
contacting the composition with the surface; and
permitting the ethyl alcohol and the water to evaporate.

9. A method for sanitizing a surface or increasing the surface's resistance to microbe transmission, comprising:
providing a composition comprising ethyl alcohol, chlorhexidine, an ester-containing moisturizer, and water;
selecting a surface to be sanitized;
contacting the composition with the surface; and
wiping the surface with a drying cloth.

10. The method according to claim 8 or claim 9 wherein the surface to be sanitized comprises a human skin area.

11. The method according to claim 8 or claim 9 wherein the surface to be sanitized comprises a person's hand.

12. The method according to claim 8 or claim 9 wherein the surface to be sanitized comprises a member selected from the group consisting of skin, a wrestling mat, a basketball, volleyball, shotput or other article of athletic equipment a computer keyboard, a chair, a hand rail, a door, a door handle, a refrigerator handle, a countertop, a gaming controller, a writing utensil and a nursery or preschool toy.

13. The method according to any of claims 8-12 wherein the ester-containing moisturizer comprises gluconolactone.

14. The method according to any of claims 8-13 wherein the composition further comprises a surfactant.

15. The method according to claim 14 wherein the surfactant comprises lauramine oxide.

16. The method according to any of claims 8-15 wherein the composition further comprises a preservative.

17. The method according to any of claims 8-16 wherein the composition further comprises colloidal silver.

18. The method according to any of claims 8-17 wherein the composition further comprises hydrogen peroxide.

19. A method for making a topical disinfectant composition for sanitizing a surface or increasing the surface's resistance to microbe transmission, comprising:
providing a first quantity of water;
dissolving a second quantity of an ester-containing moisturizer into the first quantity of water while agitating the water to provide a solution;
mixing a third quantity of ethyl alcohol into the first solution while continuing to agitate the solution to provide a mixture;
adding a fourth quantity of chlorhexidine to the mixture to provide a composition; and
continuing to agitate the composition for a period of time.

20. The method according to claim 19 wherein the water comprises distilled water.

21. The method according to claim 19 or claim 18 wherein the ester-containing moisturizer comprises gluconolactone.

22. The method according to any of claims 19-21 wherein said agitating comprises an action selected from the group consisting of stirring, shaking and combinations thereof.

23. The method according to any of claims 19-22 wherein the period of time is from about 30 seconds to about 30 minutes.

24. The method according to any of claims 19-22 wherein the period of time is from about 2 minutes to about 15 minutes.

25. The method according to any of claims 19-24 wherein the first quantity, the second quantity, the third quantity and the fourth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight and the chlorhexidine comprises from about 0.1 to about 4% of the composition by weight.

26. The method according to any of claims 19-24, further comprising diffusing a fifth quantity of a surfactant into the water or into the solution.

27. The method according to claim 22 wherein the surfactant comprises lauramine oxide.

28. The method according to any of claim 26 or claim 27 wherein the first quantity, the second quantity, the third quantity, the fourth quantity and the fifth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight and the surfactant comprises from about 0.1 to about 2% of the composition by weight.

29. The method according to any of claims 19-28, further comprising dissolving a sixth quantity of sodium benzoate into the water or into the solution.

30. The method according to claim 29 wherein the first quantity, the second quantity, the third quantity, the fourth quantity, the fifth quantity and the sixth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight, the surfactant comprises from 0 to about 2% of the composition by weight and the preservative comprises from about 0.1 to about 2% of the composition by weight.

31. The method according to any of claims 19-30, further comprising dispersing a seventh quantity of colloidal silver into the water, into the solution, into the mixture or into the composition.

32. The method according to claim 31 wherein the first quantity, the second quantity, the third quantity, the fourth quantity, the fifth quantity, the sixth quantity and the seventh quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight, the surfactant comprises from 0 to about 2% of the composition by weight, the preservative comprises from 0 to about 2% of the composition by weight and the colloidal silver comprises from about 0.1 to about 2% of the composition by weight.

33. The method according to any of claims 19-32, further comprising diffusing an eighth quantity of hydrogen peroxide into the water or into the solution.

34. The method according to claim 33 wherein the first quantity, the second quantity, the third quantity, the fourth quantity and the eighth quantity are selected such that the method produces a composition in which the water comprises from about 15 to about 98% of the composition by weight, the ester-containing moisturizer comprises from about 0.1 to about 5% of the composition by weight, the ethyl alcohol comprises from about 1 to about 85% of the composition by weight, the chlorhexidine comprises from about 0.1 to about 4% of the composition by weight and the hydrogen peroxide comprises from about 0.05 to about 30% % of the composition by weight.

35. A topical disinfectant composition for sanitizing a surface or increasing the surface's resistance to microbe transmission, the composition comprising:
hydrogen peroxide;
chlorhexidine;
an ester-containing moisturizer; and
water.

36. A method for sanitizing a surface or increasing the surface's resistance to microbe transmission, comprising:
providing a composition comprising hydrogen peroxide, chlorhexidine, an ester-containing moisturizer, and water;
selecting a surface to be sanitized;
contacting the composition with the surface; and
permitting the ethyl alcohol and the water to evaporate.

37. A method for sanitizing a surface or increasing the surface's resistance to microbe transmission, comprising:
providing a composition comprising hydrogen peroxide, chlorhexidine, an ester-containing moisturizer, and water;
selecting a surface to be sanitized;
contacting the composition with the surface; and
wiping the surface with a drying cloth.

38. A method for making a topical disinfectant composition for sanitizing a surface or increasing the surface's resistance to microbe transmission, comprising:
providing a first quantity of water;
dissolving a second quantity of an ester-containing moisturizer into the first quantity of water while agitating the water to provide a solution;
mixing a third quantity of hydrogen peroxide into the first solution while continuing to agitate the solution to provide a mixture;
adding a fourth quantity of chlorhexidine to the mixture to provide a composition; and
continuing to agitate the composition for a period of time.
